SEQUENCE LISTING (28 sequences)

SEQ ID NO: 1 - protein, 16 aa, synthetic construct
  SITE 1: note = Acetyl-azidolysine
  SITE 2: note = beta-Alanine
  SITE 6: note = (R)-2-amino-2-methyldec-9-enoic acid
  SITE 6..13: note = Olefin staple from (R)-2-amino-2-methyldec-9-enoic acid to (S)-2-amino-2-methylhept-6-enoic acid
  SITE 12: note = Cyclobutyl alanine
  SITE 13: note = (S)-2-amino-2-methylhept-6-enoic acid
  SITE 16: note = alanine amide
KXLTFXEYWAQXXAAA

SEQ ID NO: 2 - protein, 19 aa, synthetic construct
  SITE 1: note = Acetyl-azidolysine
  SITE 2: note = beta-Alanine
  SITE 6: note = (R)-2-amino-2-methyldec-9-enoic acid
  SITE 6..13: note = Olefin staple from (R)-2-amino-2-methyldec-9-enoic acid to (S)-2-amino-2-methylhept-6-enoic acid
  SITE 12: note = Cyclobutyl alanine
  SITE 13: note = (S)-2-amino-2-methylhept-6-enoic acid
  SITE 19: note = alanine amide
KXLTFXEYWAQXXAAAAAA

SEQ ID NO: 3 - protein, 19 aa, synthetic construct
  SITE 1: note = Acetyl-azidolysine
  SITE 2: note = beta-Alanine
  SITE 6: note = (R)-2-amino-2-methyldec-9-enoic acid
  SITE 6..13: note = Olefin staple from (R)-2-amino-2-methyldec-9-enoic acid to (S)-2-amino-2-methylhept-6-enoic acid
  SITE 12: note = Cyclobutyl alanine
  SITE 13: note = (S)-2-amino-2-methylhept-6-enoic acid
  SITE 19: note = D-alanine amide
KXLTFXEYWAQXXAAAAAA

SEQ ID NO: 4 - protein, 19 aa, synthetic construct
  SITE 1: note = Acetyl-azidolysine
  SITE 2: note = beta-Alanine
  SITE 6: note = (R)-2-amino-2-methyldec-9-enoic acid
  SITE 6..13: note = Olefin staple from (R)-2-amino-2-methyldec-9-enoic acid to (S)-2-amino-2-methylhept-6-enoic acid
  SITE 12: note = Cyclobutyl alanine
  SITE 13: note = (S)-2-amino-2-methylhept-6-enoic acid
  SITE 19: note = phenylalanine amide
KXLTFXEYWAQXXAAFAAF

SEQ ID NO: 5 - protein, 19 aa, synthetic construct
  SITE 1: note = Acetyl-azidolysine
  SITE 2: note = beta-Alanine
  SITE 6: note = (R)-2-amino-2-methyldec-9-enoic acid
  SITE 6..13: note = Olefin staple from (R)-2-amino-2-methyldec-9-enoic acid to (S)-2-amino-2-methylhept-6-enoic acid
  SITE 12: note = Cyclobutyl alanine
  SITE 13: note = (S)-2-amino-2-methylhept-6-enoic acid
  SITE 19: note = D-phenylalanine amide
KXLTFXEYWAQXXAAFAAF

SEQ ID NO: 6 - protein, 19 aa, synthetic construct
  SITE 1: note = Acetyl-azidolysine
  SITE 2: note = beta-Alanine
  SITE 6: note = (R)-2-amino-2-methyldec-9-enoic acid
  SITE 6..13: note = Olefin staple from (R)-2-amino-2-methyldec-9-enoic acid to (S)-2-amino-2-methylhept-6-enoic acid
  SITE 12: note = Cyclobutyl alanine
  SITE 13: note = (S)-2-amino-2-methylhept-6-enoic acid
  SITE 19: note = phenylalanine amide
KXLTFXEYWAQXXAAAAAF

SEQ ID NO: 7 - protein, 19 aa, synthetic construct
  SITE 1: note = Acetyl-azidolysine
  SITE 2: note = beta-Alanine
  SITE 6: note = (R)-2-amino-2-methyldec-9-enoic acid
  SITE 6..13: note = Olefin staple from (R)-2-amino-2-methyldec-9-enoic acid to (S)-2-amino-2-methylhept-6-enoic acid
  SITE 12: note = Cyclobutyl alanine
  SITE 13: note = (S)-2-amino-2-methylhept-6-enoic acid
  SITE 19: note = D-alanine amide
KXLTFXEYWAQXXAAFAAA

SEQ ID NO: 8 - protein, 19 aa, synthetic construct
  SITE 1: note = Acetyl-azidolysine
  SITE 2: note = beta-Alanine
  SITE 6: note = (R)-2-amino-2-methyldec-9-enoic acid
  SITE 6..13: note = Olefin staple from (R)-2-amino-2-methyldec-9-enoic acid to (S)-2-amino-2-methylhept-6-enoic acid
  SITE 12: note = Cyclobutyl alanine
  SITE 13: note = (S)-2-amino-2-methylhept-6-enoic acid
  SITE 17: note = Alpha methyl alanine
  SITE 19: note = alanine amide
KXLTFXEYWAQXXAAFAAA

SEQ ID NO: 9 - protein, 19 aa, synthetic construct
  SITE 1: note = Acetyl-azidolysine
  SITE 2: note = beta-Alanine
  SITE 6: note = (R)-2-amino-2-methyldec-9-enoic acid
  SITE 6..13: note = Olefin staple from (R)-2-amino-2-methyldec-9-enoic acid to (S)-2-amino-2-methylhept-6-enoic acid
  SITE 12: note = Cyclobutyl alanine
  SITE 13: note = (S)-2-amino-2-methylhept-6-enoic acid
  SITE 18: note = Alpha-methyl-glutamic acid
  SITE 19: note = phenylalanine amide
KXLTFXEYWAQXXAAFAEF

SEQ ID NO: 10 - protein, 19 aa, synthetic construct
  SITE 1: note = Acetyl-azidolysine
  SITE 2: note = beta-Alanine
  SITE 6: note = (R)-2-amino-2-methyldec-9-enoic acid
  SITE 6..13: note = Olefin staple from (R)-2-amino-2-methyldec-9-enoic acid to (S)-2-amino-2-methylhept-6-enoic acid
  SITE 12: note = Cyclobutyl alanine
  SITE 13: note = (S)-2-amino-2-methylhept-6-enoic acid
  SITE 19: note = D-alanine amide
KXLTFXEYWAQXXAAEAAA

SEQ ID NO: 11 - protein, 19 aa, synthetic construct
  SITE 1: note = Acetyl-azidolysine
  SITE 2: note = beta-Alanine
  SITE 6: note = (R)-2-amino-2-methyldec-9-enoic acid
  SITE 6..13: note = Olefin staple from (R)-2-amino-2-methyldec-9-enoic acid to (S)-2-amino-2-methylhept-6-enoic acid
  SITE 12: note = Cyclobutyl alanine
  SITE 13: note = (S)-2-amino-2-methylhept-6-enoic acid
  SITE 14: note = Gamma-carboxylic glutamic acid
  SITE 19: note = alanine amide
KXLTFXEYWAQXXEAAAAA

SEQ ID NO: 12 - protein, 19 aa, synthetic construct
  SITE 1: note = Acetyl-azidolysine
  SITE 2: note = beta-Alanine
  SITE 6: note = (R)-2-amino-2-methyloct-7-ynoic acid
  SITE 6..13: note = Di-alkyne staple from (R)-2-amino-2-methyloct-7-ynoic acid to (S)-2-amino-2-methylhept-6-ynoic acid
  SITE 12: note = Cyclobutyl alanine
  SITE 13: note = (S)-2-amino-2-methylhept-6-ynoic acid
  SITE 19: note = alanine amide
KXLTFXEYWAQXXAAAAAA

SEQ ID NO: 13 - protein, 19 aa, synthetic construct
  SITE 1: note = Acetyl-azidolysine
  SITE 2: note = beta-Alanine
  SITE 6: note = (R)-2-amino-2-methyloct-7-ynoic acid
  SITE 6..13: note = Di-alkyne staple from (R)-2-amino-2-methyloct-7-ynoic acid to (S)-2-amino-2-methylhept-6-ynoic acid
  SITE 12: note = Cyclobutyl alanine
  SITE 13: note = (S)-2-amino-2-methylhept-6-ynoic acid
  SITE 19: note = phenylalanine amide
KXLTFXEYWAQXXEAFAAF

SEQ ID NO: 14 - protein, 379 aa, synthetic construct
MSDKIIHSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKLEVLFQGHMHHHHHHSSGVDLGTENLYFQGMCNTNMSVPTDGAVTTSQIPASEQETLVRPKPLLLKLLKSVGAQKDTYTMKEVLFYLGQYIMTKRLYDEKQQHIVYCSNDLLGDLFGVPSFSVKEHRKIYTMIYRNLVVVNQQESSDSGTSVSEN

SEQ ID NO: 15 - protein, 9 aa, synthetic construct
  SITE 1: note = carboxyfluorescein arginine
  SITE 9: note = leucine amide
RFMDYWEGL

SEQ ID NO: 16 - protein, 6 aa, synthetic construct
AAFAAF

SEQ ID NO: 17 - protein, 6 aa, synthetic construct
AAEAAA

SEQ ID NO: 18 - protein, 22 aa, synthetic construct
  VARIANT 1: note = Any natural or non-natural L-amino acid modified with acyl or C1-12 alkyl
  SITE 2: note = beta-Alanine
  VARIANT 6: note = Any alpha-monosubstituted or alpha,alpha-disubstituted non-natural L- or D-amino acid
  CROSSLNK 6..13: note = Crosslink between alpha carbons of positions 6 and 13 via a hydrocarbon
  VARIANT 12: note = Any aliphatic natural or non-natural amino acid
  VARIANT 13: note = Any alpha-monosubstituted or alpha,alpha-disubstituted non-natural L- or D-amino acid
  VARIANT 14: note = Any natural or non-natural amino acid
  VARIANT 15: note = Any natural or non-natural amino acid
  VARIANT 16: note = Any natural or non-natural amino acid
  VARIANT 17: note = Any natural or non-natural amino acid
  VARIANT 18: note = Any natural or non-natural amino acid
  VARIANT 19: note = Any natural or non-natural amino acid and may be hydroxy or amide modified when positions 20-22 are absent
  VARIANT 20: note = Any L- or D-amino acid or absent
  VARIANT 21: note = Any L- or D-amino acid or absent
  VARIANT 22: note = Any L- or D-amino acid or absent
  VARIANT 20..22: note = This region may encompass 0-3 residues wherein the C-terminal residue may be amidated
XXLTFXEYWAQXXXXXXXXXXX

SEQ ID NO: 19 - protein, 6 aa, synthetic construct
  SITE 5: note = alpha-methyl-glutamic acid
AAFAEF

SEQ ID NO: 20 - protein, 6 aa, synthetic construct
  SITE 6: note = D-alanine
AAEAAA

SEQ ID NO: 21 - protein, 6 aa, synthetic construct
EAFAAF

SEQ ID NO: 22 - protein, 6 aa, synthetic construct
  SITE 6: note = D-alanine
AAAAAA

SEQ ID NO: 23 - protein, 5 aa, synthetic construct
  VARIANT 4: note = (R)-2-amino-2-methyldec-9-enoic acid, (R)-2-amino-2-methyloct-7-ynoic acid, or alpha-monosubstituted or alpha,alpha-disubstituted non-natural L- or D-amino acid
LTFXE

SEQ ID NO: 24 - protein, 6 aa, synthetic construct
RGDDYK

SEQ ID NO: 25 - protein, 7 aa, synthetic construct
ENLYFQS

SEQ ID NO: 26 - protein, 7 aa, synthetic construct
ENLYFQG

SEQ ID NO: 27 - protein, 17 aa, synthetic construct
  SITE 4: note = R-2-(7 prime-octenyl) alanine
  SITE 11: note = S-2-(4 prime-pentenyl) alanine
LTFXEYWAQLXAAAAAA

SEQ ID NO: 28 - protein, 6 aa, synthetic construct
AAAAAA

(19) United States
(12) Patent Application Publication
(10) Pub. No.: US 2025/0034211 A1
(43) Pub. Date: Jan. 30, 2025

(54) C-TERMINAL EXTENDED P53 ACTIVATOR CROSSLINKED PEPTIDOMIMETIC MACROCYCLES AGAINST MDM2/MDMX

(71) Applicants: Merck Sharp & Dohme LLC, Rahway, NJ (US); MSD International GmbH, Lucerne (CH); Agency for Science, Technology and Research, Singapore (SG)

(72) Inventors: Hubert Josien, Jersey City, NJ (US); Arun Chandramohan, Chennai (IN); Charles William Johannes, Cambridge, MA (US); Christopher J. Brown, Singapore (SG); Srinivasaraghavan Kannan, Bangalore (IN); Anthony William Partridge, Cambridge, MA (US); Chandra Shekhar Verma, Singapore (SG); Lin Yan, East Brunswick, NJ (US); Tsz Ying Yuen, Singapore (SG)

(73) Assignees: Merck Sharp & Dohme LLC, Rahway, NJ (US); MSD International GmbH, Lucerne (CH); Agency for Science, Technology and Research, Singapore (SG)

(21) Appl. No.: 18/712,016

(22) PCT Filed: Nov. 23, 2022
(86) PCT No.: PCT/US2022/050821

§ 371 Date: May 21, 2024

Related U.S. Application Data
(60) Provisional application No. 63/283,614, filed on Nov. 29, 2021.

(51) Int. Cl. C07K 7/56; A61K 38/00; A61K 45/06; A61P 35/00

(52) U.S. Cl. CPC C07K 7/56; A61K 45/06; A61P 35/00; A61K 38/00

(57) ABSTRACT

The crosslinked peptidomimetic macrocycles disclosed herein comprise an alkene or alkyne staple and a poly-amino acid C-terminal tail. These crosslinked peptidomimetic macrocycles have improved binding to MDM2 and MDMX (aka MDM4), are protease resistant, cell permeable without inducing membrane disruption, and intracellularly activate p53 by binding MDM2 and MDMX thereby antagonizing MDM2 and MDMX binding to p53. These peptidomimetic macrocycles may be useful in anticancer therapies, particularly in combination with chemotherapy or radiation therapy.

### REFERENCE TO SEQUENCE LISTING SUBMITTED ELECTRONICALLY

The instant application contains a Sequence Listing which has been submitted electronically in XML format and is hereby incorporated by reference in its entirety. The XML file, created on Oct. 3, 2022, is named 25345-WO-PCT-SEQLIST-03OCT2022.XML and is 71,000 bytes in size on disk.

### FIELD OF THE INVENTION

Disclosed herein are crosslinked peptidomimetic macrocycles with improved binding and/or physicochemical properties. These peptidomimetic macrocycles which can bind mouse double minute 2 (MDM2 aka E3 ubiquitin-protein ligase) and MDMX (aka MDM4) are able to permeate the cell without inducing membrane disruption, and can intracellularly activate p53 by binding MDM2 and MDMX, thereby antagonizing MDM2 and MDMX from binding p53. These macrocycles are potentially useful as anticancer therapies, particularly in combination with chemotherapy and/or radiation therapy.

### DESCRIPTION OF RELATED ART

Tumor suppressor protein p53 primarily functions as a DNA transcription factor. It is commonly abrogated in cancer and plays an important role in guarding the cell in response to various stress signals through the induction of cell cycle arrest, apoptosis, or senescence (Lane DP p53, guardian of the genome Nature. 1992 Jul. 2; 358(6381):15-6). Mechanisms that frequently result in the inactivation of p53 and tumorigenesis include increased expression of the p53-negative regulators MDM2 and MDMX (aka MDM4). Both MDM2 and MDMX attenuate p53 function by interacting directly with p53 and preventing its interaction with the relevant activation factors required for transcription, e.g., dTAF_{II} and hTAF_{II}. In addition, they are both E3 ligase components and target p53 for proteosomal mediated degradation. MDMX, unlike MDM2, has no intrinsic E3 ubiquitin ligase activity. Instead, MDMX forms heterodimeric complexes with MDM2 whereby it stimulates the ubiquitin activity of MDM2. As a result, p53 activity and protein levels are acutely suppressed by MDM2 and MDMX overexpression. Development of inhibitors to disrupt the interactions of p53 with either MDM2 or MDMX, or both, are therefore desirable as they may prevent p53 degradation and restore a p53 dependent transcriptional anti-tumor response (Arni-Schmidt, O., M. Lokshin, and C. Prives. 2016. The roles of MDM2 and MDMX in cancer. Annu. Rev. Pathol. 11:617-644; Dongsheng Pei,1,2 Yanping Zhang,1,2 and Junnian Zhengl Regulation of p53: a collaboration between MDM2 and MDMX. Oncotarget. 2012 March; 3(3): 228-235).

The structural interface of the p53-MDM2/MDMX complex is characterized by an α-helix from the N-terminal transactivation domain of p53 which binds into a hydrophobic groove on the surface of the N-terminal domain of both MDM2 and MDMX. Three hydrophobic residues, Phe19, Trp23 and Leu26, of p53 are important determinants of this interaction and project deeply into the MDM2/MDMX interaction groove [See FIG. 1]. The isolated p53 peptide is largely disordered, morphing into an α-helical conformation upon binding. There are several examples of small molecules, peptides, and biologics that mimic these interactions and compete for MDM2/MDMX binding with the release of p53 (Tisato V1, Voltan R2, Gonelli A2, Secchiero P2, Zauli G2. MDM2/X inhibitors under clinical evaluation: perspectives for the management of hematological malignancies and pediatric cancer. J Hematol Oncol. 2017 Jul. 3; 10(1):133). However, a large majority of the small molecules developed exhibit little affinity and activity against MDMX, which possesses several distinct structural differences in the p53 peptide binding groove compared to MDM2. Although several MDM2 specific molecules have entered initial clinical trials, they have largely been met with dose limiting toxicities in patients (Tisato V1, Voltan R2, Gonelli A2, Secchiero P2, Zauli G2. MDM2/X inhibitors under clinical evaluation: perspectives for the management of hematological malignancies and pediatric cancer. J Hematol Oncol. 2017 Jul. 3; 10(1):133). Overexpression of MDMX in tumors has been demonstrated to attenuate the effectiveness of MDM2 specific compounds, presumably through the maintenance of heterodimeric complexes of MDM2 and MDMX that inhibit and target p53 for proteosomal degradation. MDM2 selective inhibitors may also induce higher levels of MDMX. This highlights the importance of targeting both proteins simultaneously to achieve efficient activation of p53 to achieve an optimal therapeutic response.

Protein-protein interactions (PPIs) are central to most biological processes and are often dysregulated in diseases (Petta I., Lievens S., Libert C., Tavemier J., De Bosscher K. Modulation of Protein-Protein Interactions for the Development of Novel Therapeutics. Mol. Ther. 2015; 24:707-718; Macalino S. J. Y., Basith S., Clavio N. A. B., Chang H., Kang S., Choi S. Evolution of In Silico Strategies for Protein-Protein Interaction Drug Discovery. Molecules. 2018; 23:1963). Therefore, PPIs are attractive therapeutic targets for novel drug discovery. However, in contrast to the deep protein cavities that typically accommodate small molecules, PPI surfaces are generally large and flat, and this has contributed to a limited success in developing small molecule inhibitors for PPI targets (Scott, D. E., Bayly, A. R., Abell, C. and Skidmore, J. (2016) Small molecules, big targets: drug discovery faces the protein-protein interaction challenge. Nat. Rev. Drug Discov. 15, 533-550). The realization that 40% of all PPIs are mediated by relatively short peptide motifs gave rise to the possibility of developing peptide-based inhibitors that would compete orthosterically for the interface between ligand-target cognate partners (Nevola L, Giralt E. Modulating protein-protein interactions: the potential of peptides. Chem Commun. 2015; 51:3302-15). When taken out of the protein ligand context, such peptides may often be unstructured and intrinsically disordered, yet capable to achieve their biologically relevant conformation upon protein target binding (Nevola L, Giralt E. Modulating protein-protein interactions: the potential of peptides. Chem Commun. 2015; 51:3302-15). However, for intracellular targets, the peptide modality may be challenging due to proteolytic sensitivity, low conformational stability (yielding weak affinities and off target effects), and poor cell permeability, further limiting prosecution of intracellular targets and/or oral bioavailability (Lau, J. L.; Dunn, M. K. Therapeutic peptides: Historical perspectives, current development trends, and future directions Bioorg. Med. Chem. 2017, 26, 2700-2707; Fosgerau, K.; Hoffmann, T. Peptide therapeutics: current status and future directions Drug Discovery Today 2015, 20, 122-128; Bakail M., Ochsenbein F. Targeting protein-protein interactions, a wide open field for drug design. Comptes Rendus Chimie. 2016, 19, 19-27; A. Henninot, J. C. Collins, J. M. Nuss. The current state of peptide drug discovery: back to the future J. Med. Chem., 61 (2018), pp. 1382-1414; Morrison C., Constrained peptides' time to shine?. Nature Reviews Drug Discovery volume 17, pages 531-533 (2018); Valeur E., et al. New Modalities for Challenging Targets in Drug Discovery. Angew. Chem. Int. Ed. 2017, 56, 10294-10323; Cary, D. R.; Ohuchi, M.; Reid, P. C.; Masuya, K. Constrained Peptides in Drug Discovery and Development. Yuki Gosei Kagaku Kyokaishi 2017, 75, 1171-1178).

To address these issues, several strategies have been pursued, including macrocyclization and modifications of the peptide backbone to yield molecules with improved activities and pharmacokinetic properties as well as constraining the peptide into its biologically relevant conformation to bind its target (Lau, J. L.; Dunn, M. K. Therapeutic peptides: Historical perspectives, current development trends, and future directions Bioorg. Med. Chem. 2017, 26, 2700-2707; Fosgerau, K.; Hoffmann, T. Peptide therapeutics: current status and future directions Drug Discovery Today 2015, 20, 122-128; Bakail M., Ochsenbein F. Targeting protein-protein interactions, a wide open field for drug design. Comptes Rendus Chimie. 2016, 19, 19-27; A. Henninot, J. C. Collins, J. M. Nuss. The current state of peptide drug discovery: back to the future J. Med. Chem., 61 (2018), pp. 1382-1414; Morrison C., Constrained peptides' time to shine? Nature Reviews Drug Discovery volume 17, pages 531-533 (2018); Valeur E., et al. New Modalities for Challenging Targets in Drug Discovery. Angew. Chem. Int. Ed. 2017, 56, 10294-10323; Cary, D. R.; Ohuchi, M.; Reid, P. C.; Masuya, K. Constrained Peptides in Drug Discovery and Development. Yuki Gosei Kagaku Kyokaishi 2017, 75, 1171-1178; Vinogradov A., Macrocyclic Peptides as Drug Candidates: Recent Progress and Remaining Challenges. J. Am. Chem. Soc., 2019, 141 (10), pp 4167-4181; Sawyer, T. K. Macrocyclic a helical peptide therapeutic modality: A perspective of learnings and challenges. Bioorg. Med. Chem. 2018, 26, 2807-2815). Firstly, by biasing the peptides toward their bound conformations, entropic penalties upon binding are reduced, thus improving binding constants as well as presumably decreasing the opportunity for unwanted off-target effects. Secondly, macrocyclization may confer varying degrees of proteolytic resistance by modifying key backbone and/or side-chain structural moieties in the peptide. Thirdly, macrocyclization may enhance cell permeability, such as through increased stability of intramolecular hydrogen bonding to reduce the desolvation penalty otherwise incurred in the transport of peptides across an apolar cell membrane. Amongst the several cyclization techniques described, stapling via metathesis using a non-proteogenic amino acid such as alpha methyl alkenyl side chains has proven to be effective (Sawyer, T. K. Macrocyclic a helical peptide therapeutic modality: A perspective of leamings and challenges. Bioorg. Med. Chem. 2018, 26, 2807-2815; L. D. Walensky, G. H. Bird Hydrocarbon-stapled peptides: principles, practice, and progress J Med Chem, 57 (2014), pp. 6275-6288; Y. S. Tan, D. P. Lane, C. S. Verma Stapled peptide design: principles and roles of computation Drug Discov Today, 21 (2016), pp. 1642-1653; Ali A M., et al. Stapled Peptides Inhibitors: A New Window for Target Drug Discovery. Computational and Structural Biotechnology Journal, 2019, 17, 263-281; Klein M., Stabilized helical peptides: overview of the technologies and its impact on drug discovery. Expert Opinion on Drug Discovery. 2017, 12, 1117-1125; Lerge J et al. Stapled peptides as a new technology to investigate protein-protein interactions in human platelets. Chem Sci, 2108, 9, 4638-4643), particularly when the desired secondary structure of the peptide macrocycle is helical. Stapling requires incorporation of the appropriate non-natural amino acid precursors to be placed at appropriate locations along the peptide sequence such that they do not interfere with the binding face of the helix. The linkers can be of different types, and can span different lengths, resulting in i,i+3, i,i+4, or i,i+7 staples. Although they have largely been used to stabilize helical conformations, recent studies have also applied ring-closing metathesis (RCM) strategies to non-helical peptides (Xu W., Macrocyclized Extended Peptides: Inhibiting the Substrate-Recognition Domain of Tankyrase. J. Am. Chem. Soc., 2017, 139 (6), pp 2245-2256; Wiedmann, M M., et al. Development of Cell-Permeable, Non-Helical Constrained Peptides to Target a Key Protein-Protein Interaction in Ovarian Cancer. Angew. Chem. Int. Ed. 2017, 56, 524-529).

The stapled peptide strategy has been applied to inhibit several PPIs of therapeutic potential including, BCL-2 family-BH3 domains (L. D. Walensky, A. L. Kung, I. Escher, T. J. Malia, S. Barbuto, R. D. Wright, G. Wagner, G. L. Verdine and S. J. Korsmeyer. Activation of apoptosis in vivo by a hydrocarbon-stapled BH3 helix. Science, 2004, 305, 1466-1470; S. A. Kawamoto, A. Coleska, X. Ran, H. Yi, C. Y. Yang and S. Wang. Design of triazole-stapled BCL9 α-helical peptides to target the β-catenin/B-cell CLL/lymphoma 9 (BCL9) protein-protein interaction, J. Med. Chem., 2012, 55, 1137-1146; K. Takada, Di Zhu, G. H. Bird, K. Sukhdeo, J.-J. Zhao, M. Mani, M. Lemieux, D. E. Carrasco, J. Ryan; D. Horst, M. Fulciniti, N. C. Munshi, W. Xu, A. L. Kung, R. A. Shivdasani, L. D. Walensky and D. R. Carrasco. Targeted disruption of the BCL9/β-catenin complex inhibits oncogenic Wnt signaling. Sci. Transl. Med., 2012, 4, 148ra117), β-catenin-TCF (L Dietrich, B Rathmer, K Ewan, T Bange, S Heinrichs, T C Dale, D Schade, TN Grossmann. Cell permeable stapled peptide inhibitor of Wnt signaling that targets β-catenin protein-protein interactions. Cell Chem. Biol., 24, 958-968 (2017)), Rab-GTPase-Effector (Spiegel J, Cromm P M, Itzen A, Goody R S, Grossmann T N, Waldmann H. Direct targeting of Rab-GTPase-effector interactions. Angew Chem Int Ed Engl. 2014 Feb. 24; 53(9):2498-503), ERa-coactivator protein (Xie M., et al. Structural Basis of Inhibition of ERa-Coactivator Interaction by High-Affinity N-Terminus Isoaspartic Acid Tethered Helical Peptides. J. Med. Chem., 2017, 60 (21), pp 8731-8740), Cullin3-BTB (Paola de., et al. Cullin3-BTB interface: a novel target for stapled peptides. PLoS One. 2015 Apr. 7; 10(4):e0121149), VDR-coactivator protein (Misawa T., et al. Structural development of stapled short helical peptides as vitamin D receptor (VDR)-coactivator interaction inhibitors. Bioorg Med Chem. 2015 Mar. 1; 23(5):1055-61), eIf4E (Lama D., et al, Structural insights reveal a recognition feature for tailoring hydrocarbon stapled-peptides against the eukaryotic translation initiation factor 4E protein. Chemical Science. 2019, 10, 2489-2500), ATSP-7041 [See WO2013123266, SAH-p53-8 [Bemal et al., Cancer Cell 18: 411-422 (2010)], and p53-MDM2/MDMX (F. Bemal, A. F. Tyler, S. J. Korsmeyer, L. D. Walensky and G. L. Verdine. Reactivation of the p53 tumor suppressor pathway by a stapled p53 peptide. J. Am. Chem. Soc., 2007, 129, 2456-2457; L. K. Henchey, J. R. Porter, I. Ghosh and P. S. Arora. High specificity in protein recognition by hydrogen-bond-surrogate a-helices: selective inhibition of the p53/MDM2 complex. Chembiochem., 2010, 11, 2104-2107; C. J. Brown, S. T., Quah, J. Jong, A. M. Goh, P. C., Chiam, K. H. Khoo, M. L. Choong, M. A. Lee, L. Yurlova, K. Zolghadr, T. L. Joseph, C. S. Verma and D. P. Lane. Stapled peptides with improved potency and specificity that activate p53. ACS Chem. Biol., 2013, 8, 506-512; Y. S. Chang, B. Graves, V. Guerlavais, C. Tovar, K. Packman, T. To, K. Olson, K. Kesavan, P. Gangurde, A. Mukherjee, T. Baker, K. Darlak, C. Elkin, Z. Filipovic, F. Z Qureshi, H. Cai, P. Berry, E. Feyfant, X. E. Shi, J. Horstick, A. Annis, N. Fotouhi, T. Manning, H. Nash, L. T. Vassilev and T. K. Sawyer. Stapled a-helical peptide drug development: a potent dual inhibitor of MDM2 and MDMX for p53-dependent cancer therapy. Proc. Natl. Acad. Sci. U.S.A, 2013, 110, E3445-E3455).

In the case of p53-MDM2/MDMX, a dual selective stapled peptide ALRN-6924 (Aileron Therapeutics, Inc.) has been advanced to phase II clinical trials (A. Burgess, K. M. Chia, S. Haupt, D. Thomas, Y. Haupt and E. Lim. Clinical overview of MDM2/Xtargeted therapies. Front. Oncol., 2016, 6, 1-7; K. Kojima, J. Ishizawa and M. Andreeff. Pharmacological activation of wild-type p53 in the therapy of leukemia. Exp. Hematol., 2016, 44, 791-798; V. Tisato, R. Voltan, A. Gonelli, P. Secchiero and G. Zauli. MDM2/X inhibitors under clinical evaluation: Perspectives for the management of hematological malignancies and pediatric cancer. J. Hematol. Oncol., 2017, 10, 133). All-D configuration α-amino acid stapled peptides to provide further resistance to protease-mediated degradation has also been reported (Aronia, Pietro et al. P53 activator peptidomimetic macrocycles, WO 2020/257153).

Although the ALRN-6924 example is encouraging for the advancement of stapled peptides into the clinic, challenges yet remain. Amongst those, engineering the right set of binding elements and physicochemical properties in the molecule to provide optimal stability, solubility and cellular activity is desirable to minimize the dose required for administration to a patient. Furthermore, engineering this set of binding elements and physicochemical properties to also minimize off-target activity is desirable to reduce the risk of non-target related adverse events to the patient. The peptidomimetic macrocycles disclosed herein address these challenges and provide unexpected properties.

### BRIEF SUMMARY OF THE INVENTION

The crosslinked peptidomimetic macrocycles disclosed herein comprise an alkene or alkyne staple and a poly-amino acid C-terminal tail. These crosslinked peptidomimetic macrocycles have improved binding to MDM2 and MDMX (aka MDM4) and are protease resistant, cell permeable without inducing membrane disruption, and intracellularly activate p53 by binding MDM2 and MDMX thereby antagonizing MDM2 and MDMX binding to p53. These peptidomimetic macrocycles may be useful in anticancer therapies, particularly in combination with chemotherapy and/or radiation therapy.

### BRIEF DESCRIPTION OF THE DRAWINGS

FIG. 1 is a crystal structure of p53-MDM2 (Protein Data Bank (PDB) ID:1YCR) complex (Baek et al., JACS 134: 103-106 92012)). MDM2 is shown as surface and bound peptide is shown as a cartoon with interacting residues L-Phe19, L-Trp23, and L-Leu26 highlighted in sticks. Hydrogen bond interactions are shown as dotted lines.

FIG. 2 is a helical wheel representing azido-ATSP-7041 with 6xA tail (SEQ ID NO: 28), drawn as per https://www.biorxiv.org/content/10.1101/416347v1. Figure discloses SEQ ID NOS 27-28, respectively, in order of appearance.

FIG. 3 is a snapshot of a conformation of ATSP-7041 with AAFAAF tail (SEQ ID NO: 16)—MDM2 complex sampled during Molecular Dynamics Simulations. MDM2 is shown as surface/cartoon and the bound peptide is shown in cartoon with highlighted C-terminal residues Phe14 and Phe 17 and the hydrocarbon staple linker shown as connected sticks above the helix.

FIG. 4 is a snapshot of a conformation of ATSP-7041 with AAEAAa tail (SEQ ID NO: 17)—MDM2 complex sampled during Molecular Dynamics Simulations. MDM2 is shown as surface/cartoon and the bound peptide is shown in cartoon with highlighted interaction between Glu14 (in salmon) and Mdm2 Arg97 (in blue) side chains. The hydrocarbon staple linker is shown as connected sticks above the helix.

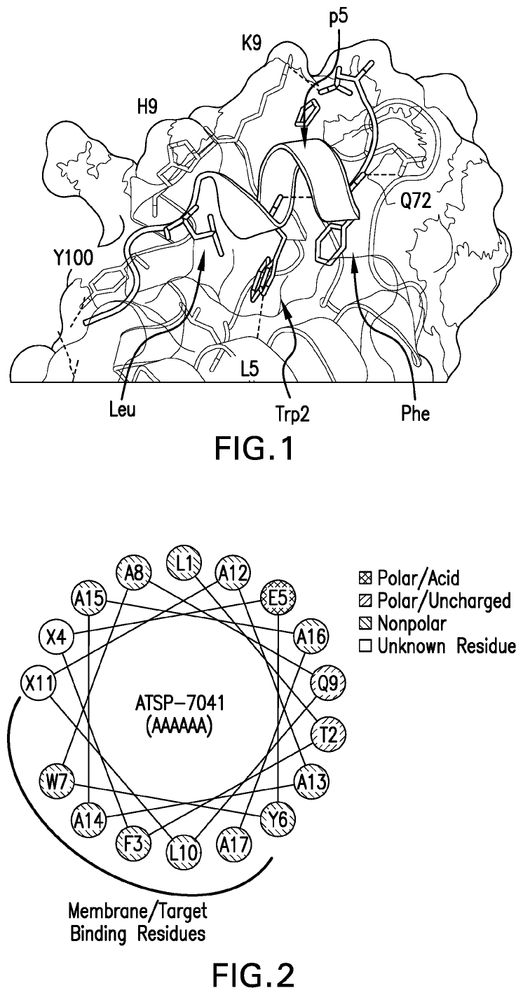

### DETAILED DESCRIPTION OF THE INVENTION

A crosslinked peptidomimetic macrocycle disclosed herein is derived from a peptidomimetic analog of a portion of human p53 protein and is represented by formula (I):

(I)
(SEQ ID NO: 18)
R₁-R₂-betaAla-L-T-F-X₁-E-Y-W-A-Q-
R₃-X₂-Z₁-Z₂-Z₃-Z₄-Z₅-Z₆-R₄,

wherein:
R₁ is selected from acyl and C₁₋₁₂ alkyl;
R₂ is a natural or non-natural L-amino acid residue;
R₃ is an aliphatic natural or non-natural amino acid residue;
R₄ is selected from —OH, —NH₂, and one to three L- or D-amino acid residues wherein the C-terminal tail is an acid or an amide group;
each of X₁ and X₂ is independently an α-monosubstituted or α,α-disubstituted non-natural L- or D-amino acid residue; and X₁ and X₂ are crosslinked from their respective alpha carbons via a hydrocarbon linkage; and
each of Z₁, Z₂, Z₃, Z₄, Z₅ and Z₆ is independently a natural or non-natural amino acid residue.

In one embodiment, R₁ is acyl. In one embodiment, R₁ is acetyl.

In one embodiment, R₁ is C₁₋₁₂ alkyl. In one embodiment, R₁ is C₁₋₄ alkyl.

In one embodiment, R₂ is selected from a Lysine residue, an azido Lysine residue and a Threonine residue.

In one embodiment, R₃ is selected from a Leucine residue and a cyclobutyl Alanine residue.

In one embodiment, R₄ is selected from —OH and —NH₂.

In one embodiment, R₄ is selected from one to three L- or D-amino acid residues wherein the C-terminal tail is an amide.

In one embodiment, R₄ is selected from is selected from —NH₂ and an L- or D-amino acid residue wherein the C-terminal tail is an amide.

In one embodiment, R₄ is —NH₂.

In one embodiment, each of X₁ and X₂ is independently an α-monosubstituted or α,α-disubstituted non-natural L- or D-amino acid residue; and X₁ and X₂ are crosslinked from their respective alpha carbons via a hydrocarbon linkage selected from an alkene and a di-alkyne. In one embodiment, the linkage is an alkene linkage. In one embodiment, the linkage is a di-alkyne linkage.

In one embodiment, each of X₁ and X₂ is independently selected from a (R)-2-amino-2-methyldec-9-enoic acid residue and an (S)-2-amino-2-methylhept-6-enoic acid residue; and X₁ and X₂ are crosslinked from their respective alpha carbons via an alkene linkage.

In one embodiment, each of X₁ and X₂ is independently selected from an (R)-2-amino-2-methyloct-7-ynoic acid residue and an (S)-2-amino-2-methylhept-6-ynoic acid residue; and X₁ and X₂ are crosslinked from their respective alpha carbons via a di-alkyne linkage.

In one embodiment, each of Z₁, Z₂, Z₄, and Z₅ is independently a natural or non-natural amino acid residue; and each of Z₃ and Z₆ is independently an alkyl or aromatic amino acid residue. In one embodiment, each of Z₃ and Z₆ is a Phenylalanine residue.

In one embodiment, one or more of Z₁, Z₂, Z₃, Z₄, Z₅ and Z₆ is independently a natural or non-natural L- or D-negatively charged amino acid residue.

In one embodiment, Z₁ is selected from a Glutamic acid residue and a gamma-carboxylic glutamic acid (Gla) residue.

In one embodiment, Z₃ is a Glutamic acid residue.

In one embodiment, Z₅ is an alpha-methyl Glutamic acid residue.

In one embodiment, the Z₁-Z₂-Z₃-Z₄-Z₅-Z₆ sequence is selected from AAFAAF (SEQ ID NO: 16), AAFA(alpha-Me-Glu)F (SEQ ID NO: 19), AAEAA(D-Ala) (SEQ ID NO: 20), EAFAAF (SEQ ID NO: 21), AAAAAA (SEQ ID NO: 28) and AAAAA(D-Ala) (SEQ ID NO: 22).

In one embodiment, the Z₁-Z₂-Z₃-Z₄-Z₅-Z₆ sequence is selected from AAFAAF (SEQ ID NO: 16), AAFA(alpha-Me-Glu)F (SEQ ID NO: 19), EAFAAF (SEQ ID NO: 21) and AAAAA(D-Ala) (SEQ ID NO: 22).

In one embodiment of the crosslinked peptidomimetic macrocycle of formula (I):
R₁ is acetyl;
R₂ is selected from a Lysine residue and an azido Lysine residue;
R₃ is a cyclobutyl Alanine residue;
R₄ is —NH₂;
each of X₁ and X₂ is independently an α-monosubstituted or α,α-disubstituted non-natural L- or D-amino acid residue; and X₁ and X₂ are crosslinked from their respective alpha carbons via an alkene or a di-alkyne hydrocarbon linkage; and
the Z₁-Z₂-Z₃-Z₄-Z₅-Z₆ sequence is selected from AAFAAF (SEQ ID NO: 16), AAFA(alpha-Me-Glu)F (SEQ ID NO: 19), AAEAA(D-Ala) (SEQ ID NO: 20), EAFAAF (SEQ ID NO: 21), AAAAAA (SEQ ID NO: 28) and AAAAA(D-Ala) (SEQ ID NO: 22).

In one embodiment of the crosslinked peptidomimetic macrocycle of formula (I):
R₁ is acetyl;
R₂ is selected from a Lysine residue and an azido Lysine residue;
R₃ is a cyclobutyl Alanine residue;
R₄ is —NH₂;
each of X₁ and X₂ is independently selected from an (R)-2-amino-2-methyldec-9-enoic acid residue and an (S)-2-amino-2-methylhept-6-enoic acid residue; and X₁ and X₂ are crosslinked from their respective alpha carbons via an alkene linkage; and
the Z₁-Z₂-Z₃-Z₄-Z₅-Z₆ sequence is selected from AAFAAF (SEQ ID NO: 16), AAFA(alpha-Me-Glu)F (SEQ ID NO: 19), EAFAAF (SEQ ID NO: 21) and AAAAA(D-Ala) (SEQ ID NO: 22).

In one embodiment of the crosslinked peptidomimetic macrocycle of formula (I):
R₁ is acetyl;
R₂ is selected from a Lysine residue and an azido Lysine residue;
R₃ is a cyclobutyl Alanine residue;
R₄ is —NH₂;
each of X₁ and X₂ is independently selected from an (R)-2-amino-2-methyloct-7-ynoic acid residue and an (S)-2-amino-2-methylhept-6-ynoic acid residue; and X₁ and X₂ are crosslinked from their respective alpha carbons via a di-alkyne linkage; and
the Z₁-Z₂-Z₃-Z₄-Z₅-Z₆ sequence is selected from AAFAAF (SEQ ID NO: 16), AAFA(alpha-Me-Glu)F (SEQ ID NO: 19), EAFAAF (SEQ ID NO: 21) and AAAAA(D-Ala) (SEQ ID NO: 22).

The Z₁-Z₂-Z₃-Z₄-Z₅-Z₆ sequence disclosed herein can improve binding to MDM2 and/or MDMX. In one embodiment, the Z₁-Z₂-Z₃-Z₄-Z₅-Z₆ sequence is modified to further improve cellular activity in the p53 cellular reporter gene assay.

The crosslinked peptidomimetic macrocycles disclosed herein bind MDM2 and MDMX, are cell permeable without inducing detectable disruption to the cell membrane, and activate p53 intracellularly. They interfere with the binding of p53 to MDM2 and/or of p53 to MDMX, thereby liberating functional p53 and inhibiting its destruction.

The crosslinked peptidomimetic macrocycles described herein may be useful for treating cancers and other disorders characterized by an undesirably low level or a low activity of p53, and/or for treating cancers and other disorders characterized by an undesirably high level of activity of MDM2 or MDMX. They may also be useful for treating a disorder associated with disrupted regulation of the p53 transcriptional pathway, leading to conditions of excess cell survival and proliferation such as cancer and autoimmunity, in addition to conditions of inappropriate cell cycle arrest and apoptosis such as neurodegeneration and immunodeficiencies.

These crosslinked peptidomimetic macrocycles contain two modified amino acids that together form an intramolecular cross-link that stabilizes the alpha-helical secondary structure of a portion of the peptides that antagonizes the binding of p53 to MDM2 and/or MDMX. The cross-linking is referred to as a “staple” and the crosslinked peptide as a “stapled peptide”.

In one embodiment of the crosslinked peptidomimetic macrocycles, each α-monosubstituted or α,α-disubstituted amino acid residue at position X₁ or X₂ comprises one or two α-carbon-linked reactive groups wherein the reactive group of a first α-monosubstituted or α,α-disubstituted amino acid residue is capable of reacting with the reactive group of a second α-monosubstituted or α,α-disubstituted amino acid residue to form a cross-linker. In one embodiment, the reactive group comprises a terminal olefin group. In another embodiment, the reactive group comprises a terminal alkyne group.

In one embodiment of the crosslinked peptidomimetic macrocycles, the non-natural amino acid residue at position X₁ is an (R)-2-amino-2-methyldec-9-enoic acid residue; at position X₂ is an (S)-2-amino-2-methylhept-6-enoic acid residue; and the staple is an olefin obtained through ring-closing metathesis.

In one embodiment of the crosslinked peptidomimetic macrocycles, the non-natural amino acid at position X₁ is an (R)-2-amino-2-methyloct-7-ynoic acid residue; at position X₂ is an (S)-2-amino-2-methylhept-6-ynoic acid residue; and the staple is an olefin obtained through Glaser coupling.

In one embodiment, a crosslinked peptidomimetic macrocycle comprises the amino acid sequence set forth in SEQ ID NO: 4 represented by formula (IV):

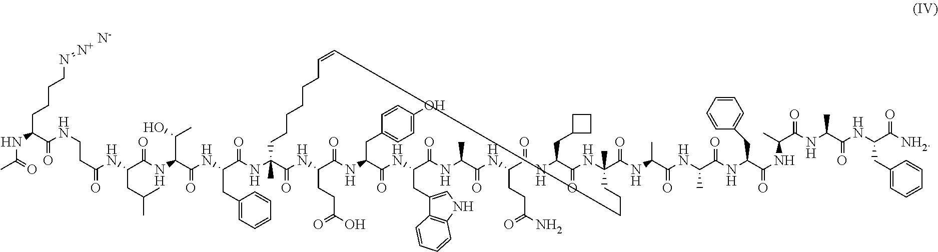

In one embodiment, a crosslinked peptidomimetic macrocycle comprises the amino acid sequence set forth in SEQ ID NO: 9 represented by the formula (IX):

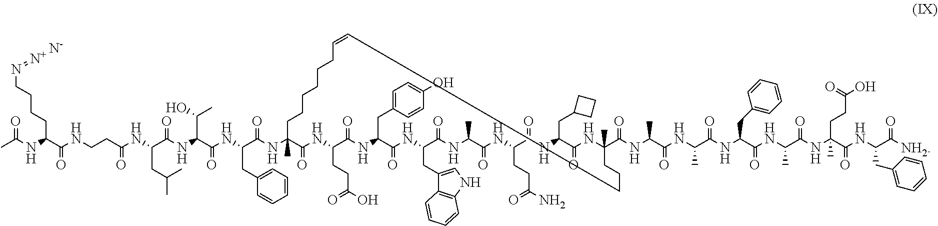

In one embodiment, a crosslinked peptidomimetic macrocycle comprises the amino acid sequence set forth in SEQ ID NO: 10 represented by the formula (X):

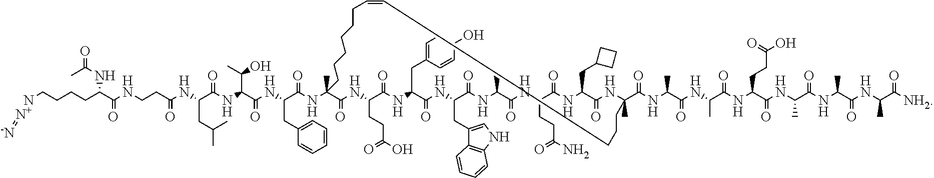

In one embodiment, a crosslinked peptidomimetic macrocycle comprises the amino acid sequence set forth in SEQ ID NO: 13 represented by formula (XIII):

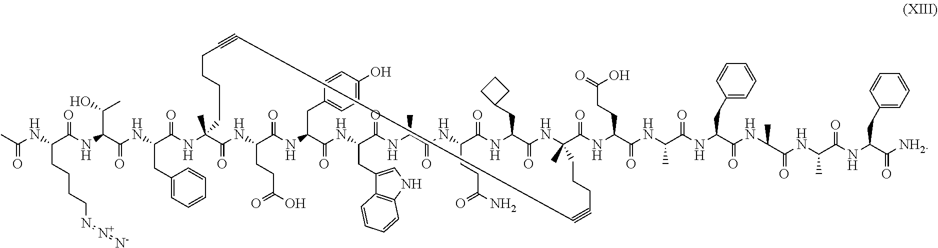 [SMILES: CC(=O)N[C@@H](CCCCN=[N+]=[N-])C(=O)N[C@H](C(=O)N[C@@H](Cc1ccccc1)C(=O)N[C@]1(C)CCCCC#CC#CCCC[C@@](C)(C(=O)N[C@@H](CCC(=O)O)C(=O)N[C@@H](C)C(=O)N[C@@H](Cc2ccccc2)C(=O)N[C@H](C)C(=O)N[C@@H](C)C(=O)N[C@@H](Cc2ccccc2)C(N)=O)NC(=O)[C@H](CC2CCC2)NC(=O)[C@H](CCC(N)=O)NC(=O)[C@H](C)NC(=O)[C@H](Cc2c[nH]c3ccccc23)NC(=O)[C@H](Cc2ccc(O)cc2)NC(=O)[C@H](CCC(=O)O)NC1=O)[C@@H](C)O]

In one embodiment, a crosslinked peptidomimetic macrocycle binds both MDM2 and MDMX, is cell permeable with no detectable disruption of the cell membrane as determined by a lactate dehydrogenase (LDH) release assay, and activates p53 intracellularly.

In one embodiment, a crosslinked peptidomimetic macrocycle binds both MDM2 and MDMX, activates p53 intracellularly, and is selective in a counter screen assay.

In one embodiment, a crosslinked peptidomimetic macrocycle binds both MDM2 and MDMX, is cell permeable with no detectable disruption of the cell membrane as determined by a lactate dehydrogenase (LDH) release assay, activates p53 intracellularly, and is selective in a counter screen assay.

The amino acid sequences of Reference Macrocycle 1 (SEQ ID NO 1) and Macrocycles 2-13 (SEQ ID NOS. 2-13) are listed in Table 1.

TABLE 1
Sequences of Reference Macrocycle 1 and Macrocycles 2-13

| SEQ ID NO | Sequence |
|---|---|
| 1 | Ac-K(N3)-betaAla-L-T-F-X1a-E-Y-W-A-Q-Cba-X2a-AAA-NH₂ |
| | (olefin staple from X1a to X2a) |
| 2 | Ac-K(N3)-betaAla-L-T-F-X1a-E-Y-W-A-Q-Cba-X2a-AAAAAA-NH₂ |
| | (olefin staple from X1a to X2a) |
| 3 | Ac-K(N3)-betaAla-L-T-F-X1a-E-Y-W-A-Q-Cba-X2a-AAAAA-(D- |
| | Ala)-NH₂(olefin staple from X1a to X2a) |
| 4 | Ac-K(N3)-betaAla-L-T-F-X1a-E-Y-W-A-Q-Cba-X2a-AAFAAF-NH₂ |
| | (olefin staple from X1a to X2a) |
| 5 | Ac-K(N3)-betaAla-L-T-F-X1a-E-Y-W-A-Q-Cba-X2a-AAFAA-(D- |
| | Phe)-NH₂(olefin staple from X1a to X2a) |
| 6 | Ac-K(N3)-betaAla-L-T-F-X1a-E-Y-W-A-Q-Cba-X2a-AAAAAF-NH₂ |
| | (olefin staple from X1a to X2a) |
| 7 | Ac-K(N3)-betaAla-L-T-F-X1a-E-Y-W-A-Q-Cba-X2a-AAFAA-(D- |
| | Ala)-NH₂(olefin staple from X1a to X2a) |
| 8 | Ac-K(N3)-betaAla-L-T-F-X1a-E-Y-W-A-Q-Cba-X2a-AAF-Aib-AA- |
| | NH₂(olefin staple from X1a to X2a) |
| 9 | Ac-K(N3)-betaAla-L-T-F-X1a-E-Y-W-A-Q-Cba-X2a-AAFA-(alpha- |
| | methyl-Glu)-F-NH₂(olefin staple from X1a to X2a) |
| 10 | Ac-K(N3)-betaAla-L-T-F-X1a-E-Y-W-A-Q-Cba-X2a-AAEAA-(D- |
| | Ala)-NH₂(olefin staple from X1a to X2a) |
| 11 | Ac-K(N3)-betaAla-L-T-F-X1a-E-Y-W-A-Q-Cba-X2a-(Gla)AAAAA- |
| | NH₂(olefin staple from X1a to X2a) |
| 12 | Ac-K(N3)-betaAla-L-T-F-X1b-E-Y-W-A-Q-Cba-X2b-AAAAAA-NH₂ |
| | (di-alkyne staple from X1b to X2b) |
| 13 | Ac-K(N3)-betaAla-L-T-F-X1b-E-Y-W-A-Q-Cba-X2b-EAFAAF-NH₂ |
| | (di-alkyne staple from X1b to X2b) |

X1a is an (R)-2-amino-2-methyldec-9-enoic acid residue
X2a is an (S)-2-amino-2-methylhept-6-enoic acid residue
X1b is an (R)-2-amino-2-methyloct-7-ynoic acid residue
X2b is an (S)-2-amino-2-methylhept-6-ynoic acid residue
Cba is a cyclobutyl alanine residue
Aib is an alpha methyl alanine residue
Gla is a gamma-carboxylic glutamic acid residue

Also disclosed herein is a method of modulating the activity of p53 and/or MDM2 and/or MDMX in a subject comprising administering to the subject a crosslinked peptidomimetic macrocycle of structural formula (I), including any one of the aforementioned peptidomimetic macrocycles.

Further disclosed herein is a method of antagonizing the interaction between p53 and MDM2 and/or between p53 and MDMX proteins in a subject comprising administering to the subject a crosslinked peptidomimetic macrocycle of structural formula (I), including any one of the aforementioned peptidomimetic macrocycles.

Further disclosed herein is a crosslinked peptidomimetic macrocycle of structural formula (I), including any one of the aforementioned peptidomimetic macrocycles for the treatment of cancer. For example, a method for treating cancer in a subject having a cancer comprises administering to the subject any one of the aforementioned crosslinked peptidomimetic macrocycles.

In one embodiment, the cancer is selected from melanoma, non-small cell lung cancer, head and neck cancer, urothelial cancer, breast cancer, gastrointestinal cancer, multiple myeloma, hepatocellular cancer, non-Hodgkin lymphoma, renal cancer, Hodgkin lymphoma, mesothelioma, ovarian cancer, small cell lung cancer, esophageal cancer, anal cancer, biliary tract cancer, colorectal cancer, cervical cancer, thyroid cancer, salivary cancer, pancreatic cancer, bronchus cancer, prostate cancer, pancreatic cancer, stomach cancer, ovarian cancer, urinary bladder cancer, brain or central nervous system cancer, peripheral nervous system cancer, uterine or endometrial cancer, cancer of the oral cavity or pharynx, liver cancer, kidney cancer, testicular cancer, biliary tract cancer, small bowel or appendix cancer, adrenal gland cancer, osteosarcoma, chondrosarcoma, and cancer of hematological tissues.

Further disclosed herein is a combination therapy for treating cancer comprising administering to a subject a therapeutically effective amount of a crosslinked peptidomimetic macrocycle of structural formula (I), including any one of the aforementioned peptidomimetic macrocycles and a therapeutically effective dose of a chemotherapy agent or radiation. In one embodiment, the chemotherapy agent or radiation is administered to the subject followed by administration of the peptidomimetic macrocycle; the peptidomimetic macrocycle is administered to the subject followed by administration of the chemotherapy agent or radiation; or the chemotherapy agent or radiation is administered to the subject simultaneously with administration of the peptidomimetic macrocycle. Thus, in one embodiment, a combination therapy for the treatment of a cancer comprises a therapeutically effective amount of a crosslinked peptidomimetic macrocycle of any one of the aforementioned peptidomimetic macrocycles and a therapeutically dose of a chemotherapy agent or radiation.

In one embodiment, a combination therapy for treating cancer comprises administering to a subject a therapeutically effective amount of a crosslinked peptidomimetic macrocycle of structural formula (I), including any one of the aforementioned peptidomimetic macrocycles and a therapeutically effective amount of a checkpoint inhibitor. In a particular aspect, the checkpoint inhibitor is an anti-PD1 antibody or an anti-PD-L1 antibody. In a further aspect, the therapy further comprises administering to the subject a therapeutically effective dose of a chemotherapy agent or radiation.

In one embodiment, a treatment of cancer comprises administering to a subject having the cancer a vector comprising a nucleic acid molecule encoding a wild-type p53 protein or p53 variant with transcriptional activation activity followed by one or more administrations of a therapeutically effective amount of a crosslinked peptidomimetic macrocycle of structural formula (I), including any one of the aforementioned peptidomimetic macrocycles. In one embodiment, the vector is a plasmid, a retrovirus, adenovirus, or adeno-associated virus. In one embodiment, the subject is administered a chemotherapy or radiation treatment prior to administering the vector to the subject or subsequent to administering the vector to the subject. In one embodiment, the therapy comprises administering to the subject a checkpoint inhibitor prior to administering the vector to the subject or subsequent to administering the vector to the subject. The checkpoint inhibitor may be administered prior to administering the chemotherapy or radiation treatment to the subject or subsequent to administering the chemotherapy or radiation treatment to the subject.

In one embodiment, the chemotherapy agent is selected from actinomycin, all-trans retinoic acid, alitretinoin, azacitidine, azathioprine, bexarotene, bleomycin, bortezomib, carmofur, carboplatin, capecitabine, cisplatin, chloranbucil, cyclophosphamide, cytarabine, dacarbazine, daunorubicin, docetaxel, doxifluridine, doxorubicin, epirubicin, epothilone, etoposide, fluorouracil, gemcitabin, hydroxyurea, idarubicin, imatinib, ixabepilone, irinotecan, mechlorethamine, melphalan, mercaptopurine, methotrexate, mitoxantrone, nitrosoureas, oxaliplatin, paclitaxel, pemetrexed, romidepsin, tegafur, temozolomide (oral dacarbazine), teniposide, tioguanine, topotecan, utidelone, valrubicin, vemurafenib, vinblastine, vincristine, vindesine, vinorelbine, and vorinostat.

In one embodiment, disclosed herein is a pharmaceutical composition comprising any one of the aforementioned peptidomimetic macrocycles and a pharmaceutically acceptable carrier or excipient. In one embodiment, the peptidomimetic macrocycle is selected from SEQ ID NO: 4, SEQ ID NO: 9, SEQ ID NO: 10, and SEQ ID NO: 13.

### Definitions

“Acyl” refers to a class of organic radicals, RCO—, formed by the removal of a hydroxyl group from a carboxylic acid. In one embodiment, R is C₁₋₁₂ alkyl. In one embodiment, R is C₁₋₄ alkyl. In one embodiment, an acyl is an acetyl (i.e., R is a methyl).

“Administer” and “administering” are used to mean introducing at least one peptidomimetic macrocycle, or a pharmaceutical composition comprising at least one peptidomimetic macrocycle, into a subject. When administration is for the purpose of treatment, the substance is provided at, or after the diagnosis of an abnormal cell growth, such as a tumor. The therapeutic administration of this substance serves to inhibit cell growth of the tumor or abnormal cell growth.

“Alkyl” refers to both branched- and straight-chain saturated aliphatic hydrocarbon groups of 1 to 18 carbon atoms (C₁₋₁₈ alkyl), or more specifically, 1 to 12 carbon atoms (C₁₋₁₂ alkyl), or even more specifically, 1 to 4 carbon atoms (C₁₋₄ alkyl). In one embodiment, an alkyl is a methyl.

The term “α-amino acid” or simply “amino acid” refers to a molecule containing both an amino group and a carboxyl group bound to a carbon, which is designated the α-carbon, attached to a side chain (R group) and a hydrogen atom and may be represented by the formula shown for (R) and (S) α-amino acids:

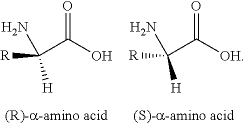

In general, L-amino acids have an (S) configuration except for cysteine, which has an (R) configuration, and glycine, which is achiral. Suitable α-amino acids for the all-D configuration peptides disclosed herein include only the D-isomers of the naturally occurring amino acids and analogs thereof, as well as non-naturally occurring amino acids prepared by organic synthesis or other metabolic routes except for α,α-disubstituted amino acids, which may be L, D, or achiral. Unless the context specifically indicates otherwise, the term amino acid, as used herein, is intended to include amino acid analogs. As used herein, D amino acids are denoted by the superscript “D” (e.g., ^{D}Leu) and L amino acids by “L” (e.g., L-Leu) or no L identifier (e.g., Leu).

The term “α,α-disubstituted amino acid” refers to a molecule or moiety containing both an amino group and a carboxyl group bound to the α-carbon that is attached to two natural or non-natural amino acid side chains, or combination thereof. Exemplary α,α-disubstituted amino acids are shown below. These α,α-disubstituted amino acids comprise a side chain with a terminal olefinic reactive group.

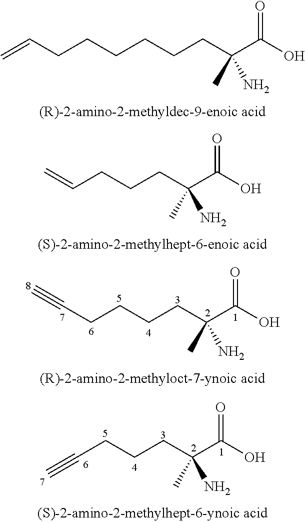

“Amino acid analog” or “non-natural amino acid” refers to a molecule which is structurally similar to an amino acid and which can be substituted for an amino acid in the formation of a peptidomimetic macrocycle. Amino acid analogs include, without limitation, compounds which are structurally identical to an amino acid, as defined herein, except for the inclusion of one or more additional methylene groups between the amino and carboxyl group (e.g., α-amino, β-carboxy acids), or for the substitution of the amino or carboxy group by a similarly reactive group (e.g., substitution of the primary amine with a secondary or tertiary amine, or substitution or the carboxy group with an ester).

“Amino acid side chain” refers to a moiety attached to the α-carbon in an amino acid. For example, the amino acid side chain for alanine is methyl, the amino acid side chain for phenylalanine is phenylmethyl, the amino acid side chain for cysteine is thiomethyl, the amino acid side chain for aspartate is carboxymethyl, the amino acid side chain for tyrosine is 4-hydroxyphenylmethyl, etc. Other non-naturally occurring amino acid side chains are also included, for example, those that occur in nature (e.g., an amino acid metabolite) or those that are made synthetically (e.g., an α,α-disubstituted amino acid).

“Capping group” refers to the chemical moiety occurring at either the carboxy or amino terminus of the polypeptide chain of the subject peptidomimetic macrocycle. The capping group of a carboxy terminus includes an unmodified carboxylic acid (i.e., —COOH) or a carboxylic acid with a substituent. For example, the carboxy terminus can be substituted with an amino group to yield a carboxamide at the C-terminus (i.e., —CONH₂). Various substituents include but are not limited to primary and secondary amines, including pegylated secondary amines. The capping group of an amino terminus includes an unmodified amine (i.e. —NH₂) or an amine with a substituent. For example, the amino terminus can be substituted with an acyl group to yield a carboxamide at the N-terminus. Various substituents include but are not limited to substituted acyl groups, including C₁-C₆ carbonyls, C₇-C₃₀ carbonyls, and pegylated carbamates.

“Co-administer” means that each of at least two different biologically active compounds are administered to a subject during a time frame wherein the respective periods of biological activity overlap. Thus, the term includes sequential as well as co-extensive administration. When co-administration is used, the routes of administration need not be the same. The biologically active compounds include peptidomimetic macrocycles, as well as other compounds useful in treating cancer, including but not limited to agents such as vinca alkaloids, nucleic acid inhibitors, platinum agents, interleukin-2, interferons, alkylating agents, antimetabolites, corticosteroids, DNA intercalating agents, anthracyclines, and ureas. Examples of specific agents in addition to those exemplified herein, include hydroxyurea, 5-fluorouracil, anthramycin, asparaginase, bleomycin, dactinomycin, dacabazine, cytarabine, busulfan, thiotepa, lomustine, mechlorehamine, cyclophosphamide, melphalan, mechlorethamine, chlorambucil, carmustine, 6-thioguanine, methotrexate, etc. The skilled artisan will understand that two different peptidomimetic macrocycles may be co-administered to a subject, or that a peptidomimetic macrocycle and an agent, such as one of the agents provided above, may be co-administered to a subject.

“Combination therapy” as used herein refers to treatment of a human or animal individual comprising administering a first therapeutic agent and a second therapeutic agent consecutively or concurrently to the individual. In general, the first and second therapeutic agents are administered to the individual separately and not as a mixture; however, there may be embodiments where the first and second therapeutic agents are mixed prior to administration.

“Conservative substitution” as used herein refers to substitutions of amino acids with other amino acids having similar characteristics (e.g. charge, side-chain size, hydrophobicity/hydrophilicity, backbone conformation and rigidity, etc.), such that the changes can frequently be made without altering the biological activity of the protein. Those of skill in this art recognize that, in general, single amino acid substitutions in non-essential regions of a polypeptide do not substantially alter biological activity (see, e.g., Watson et al. Molecular Biology of the Gene, The Benjamin/Cummings Pub. Co., p. 224 (4th Ed.) (1987)). In addition, substitutions of structurally or functionally similar amino acids are less likely to disrupt biological activity. Exemplary conservative substitutions are set forth in Table 2.

TABLE 2

| Original | Conservative | Original | Conservative |
|---|---|---|---|
| residue | substitution | residue | substitution |
| Ala (A) | Gly; Ser | Leu (L) | Ile; Val |
| Arg (R) | Lys; His | Lys (K) | Arg; His |
| Asn (N) | Gln; His | Met (M) | Leu; Ile; Tyr |
| Asp (D) | Glu; Asn | Phe (F) | Tyr; Met; Leu |
| Cys (C) | Ser; Ala | Pro (P) | Ala |
| Gln (Q) | Asn | Ser (S) | Thr |
| Glu (E) | Asp; Gln | Thr (T) | Ser |
| Gly (G) | Ala | Trp (W) | Tyr; Phe |
| His (H) | Asn; Gln | Tyr (Y) | Trp; Phe |
| Ile (I) | Leu; Val | Val (V) | Ile; Leu |

“Dose”, “dosage”, “unit dose”, “unit dosage”, “effective dose” and related terms refer to physically discrete units that contain a predetermined quantity of active ingredient (e.g., peptidomimetic macrocycle) calculated to produce a desired therapeutic effect (e.g., death of cancer cells). These terms are synonymous with the therapeutically effective amounts and amounts sufficient to achieve the stated goals of the methods disclosed herein.

“Helical stability” refers to the maintenance of α-helical structure by the staples or stitch of a peptidomimetic macrocycle as measured by circular dichroism or NMR. For example, in some embodiments, the peptidomimetic macrocycles disclosed herein exhibit at least a 1.25, 1.5, 1.75 or 2-fold increase in α-helicity as determined by circular dichroism compared to a corresponding un-crosslinked macrocycle.

“Macrocycle” refers to a molecule having a chemical structure including a ring or cycle formed by at least nine covalently bonded atoms.

“Macrocyclization reagent” or “macrocycle-forming reagent” as used herein refers to any reagent which may be used to prepare a peptidomimetic macrocycle of the invention by mediating the reaction between two reactive groups. Reactive groups may be, for example, an azide and alkyne, in which case macrocyclization reagents include, without limitation, Cu reagents such as reagents which provide a reactive Cu(I) species, such as CuBr, CuI or CuOTf, as well as Cu(II) salts such as Cu(CO₂CH₃)₂, CuSO₄, and CuCl₂ that can be converted in situ to an active Cu(I) reagent by the addition of a reducing agent such as ascorbic acid or sodium ascorbate.

Macrocyclization reagents may additionally include, for example, Ru reagents known in the art such as Cp*RuCl(PPh₃)₂, [Cp*RuCl]₄ or other Ru reagents which may provide a reactive Ru(II) species. In other cases, the reactive groups are terminal olefins. In such embodiments, the macrocyclization reagents or macrocycle-forming reagents are metathesis catalysts including, but not limited to, stabilized, late transition metal carbene complex catalysts such as Group VIII transition metal carbene catalysts. For example, such catalysts are Ru and Os metal centers having a +2 oxidation state, an electron count of 16 and pentacoordinated. Additional catalysts are disclosed in Grubbs et al., “Ring Closing Metathesis and Related Processes in Organic Synthesis” Acc. Chem. Res. 1995, 28, 446-452, and U.S. Pat. No. 5,811,515. In yet other cases, the reactive groups are thiol groups. In such embodiments, the macrocyclization reagent is, for example, a linker functionalized with two thiol-reactive groups such as halogen groups.

“MDM2” refers to the mouse double minute 2 protein also known as E3 ubiquitin-protein ligase. MDM2 is a protein that in humans is encoded by the MDM2 gene. MDM2 protein is an important negative regulator of the p53 tumor suppressor. MDM2 protein functions both as an E3 ubiquitin ligase that recognizes the N-terminal trans-activation domain (TAD) of the p53 tumor suppressor and as an inhibitor of p53 transcriptional activation. As used herein, the term MDM2 refers to the human homolog. See GenBank Accession No.: 228952; GI:228952.

“MDMX” or “MDM4” refers to mouse double minute X or 4, a protein that shows significant structural similarity to MDM2. MDMX or MDM4 interacts with p53 via a binding domain located in the N-terminal region of the MDMX or MDM4 protein. As used herein, the term MDMX or MDM4 refers to the same human homolog. See GenBank Accession No.: 88702791; GI:88702791.

“Member” as used herein in conjunction with macrocycles or macrocycle-forming linkers refers to the atoms that form or can form the macrocycle, and excludes substituent or side chain atoms. By analogy, cyclodecane, 1,2-difluoro-decane and 1,3-dimethyl cyclodecane are all considered ten-membered macrocycles as the hydrogen or fluoro substituents or methyl side chains do not participate in forming the macrocycle.

“Naturally occurring amino acid” refers to any one of the twenty amino acids commonly found in peptides synthesized in nature, and known by the one letter abbreviations A, R, N, C, D, Q, E, G, H, I, L, K, M, F, P, S, T, W, Y and V.

“Non-essential” amino acid residue is a residue that can be altered from the wild-type sequence of a polypeptide without abolishing or substantially altering the polypeptide's essential biological or biochemical activity (e.g., receptor binding or activation). An “essential” amino acid residue is a residue that, when altered from the wild-type sequence of the polypeptide, results in abolishing or substantially abolishing the polypeptide's essential biological or biochemical activity.

“Peptidomimetic macrocycle” or “crosslinked polypeptide” refers to a compound comprising a plurality of amino acid residues joined by a plurality of peptide bonds and at least one macrocycle-forming linker, which forms a macrocycle between a first naturally occurring or non-naturally occurring amino acid residue (or analog) and a second naturally occurring or non-naturally occurring amino acid residue (or analog) within the same molecule. The peptidomimetic macrocycle include embodiments where the macrocycle-forming linker connects the α-carbon of the first amino acid residue (or analog) to the α-carbon of the second amino acid residue (or analog). Peptidomimetic macrocycles optionally include one or more non-peptide bonds between one or more amino acid residues and/or amino acid analog residues, and optionally include one or more non-naturally occurring amino acid residues or amino acid analog residues in addition to any which form the macrocycle. A “corresponding non-crosslinked polypeptide” when referred to in the context of a peptidomimetic macrocycle is understood to relate to a polypeptide of the same amino acid sequence as the peptidomimetic macrocycle except for those amino acids involved in the staple or stitch crosslinks.

Unless otherwise stated, peptidomimetic macrocycles and structures referred to herein are also meant to include peptidomimetic macrocycles which differ only in the presence of one or more isotopically enriched atoms. For example, peptidomimetic macrocycles having the present structures wherein hydrogen is replaced by deuterium or tritium, or wherein carbon atom is replaced by ¹³C- or ¹⁴C-enriched carbon, or wherein a carbon atom is replaced by silicon, are within the scope of this disclosure. The peptidomimetic macrocycles disclosed herein may also contain non-natural proportions of atomic isotopes at one or more of atoms that constitute such peptidomimetic macrocycles. For example, the peptidomimetic macrocycles may be radiolabeled with radioactive isotopes, such as for example tritium (³H), iodine-125 (¹²⁵I) or carbon-14 (¹⁴C). All isotopic variations of the peptidomimetic macrocycles of the present invention, whether radioactive or not, are encompassed within the scope of the present disclosure.

“Pharmaceutically acceptable derivative” means any pharmaceutically acceptable salt, ester, salt of an ester, pro-drug or other derivative of a peptidomimetic macrocycle disclosed herein, which upon administration to an individual, is capable of providing (directly or indirectly) a peptidomimetic macrocycle disclosed herein. Particularly favored pharmaceutically acceptable derivatives are those that increase the bioavailability of the peptidomimetic macrocycle disclosed herein when administered to an individual (e.g., by increasing absorption into the blood of an orally administered peptidomimetic macrocycle disclosed herein) or which increases delivery of the active compound to a biological compartment (e.g., the brain or lymphatic system) relative to the parent species. Some pharmaceutically acceptable derivatives include a chemical group which increases aqueous solubility or active transport across the gastrointestinal mucosa.

“Polypeptide” encompasses two or more naturally or non-naturally occurring amino acids joined by a covalent bond (e.g., an amide bond). Polypeptides as described herein include full length proteins (e.g., fully processed proteins) as well as shorter amino acid sequences (e.g., fragments of naturally occurring proteins or synthetic polypeptide fragments).

“Stability” refers to the maintenance of a defined secondary structure in solution by a peptidomimetic macrocycle of the invention as measured by circular dichroism, NMR or another biophysical measure, or resistance to proteolytic degradation in vitro or in vivo. Non-limiting examples of secondary structures contemplated in this invention are α-helices, β-turns, and β-pleated sheets.

“Therapeutically effective amount” or “Therapeutically effective dose” as used herein refers to a quantity of a specific substance sufficient to achieve a desired effect in a subject being treated. For instance, this may be the amount of peptidomimetic macrocycle of the present invention necessary to activate p53 by inhibiting its binding to MDM2 and MDMX. It may also refer to the amount or dose of a chemotherapy agent or radiation administered to a subject that has cancer that is commonly administered to the subject to treat the cancer.

“Treat” or “treating” as used herein means to administer a therapeutic agent, such as a composition containing any of peptidomimetic macrocycles of the present invention, internally or externally to a subject or patient having one or more disease symptoms, or being suspected of having a disease, for which the agent has therapeutic activity or prophylactic activity. Typically, the agent is administered in an amount effective to alleviate one or more disease symptoms in the treated subject or population, whether by inducing the regression of or inhibiting the progression of such symptom(s) by any clinically measurable degree. The amount of a therapeutic agent that is effective to alleviate any particular disease symptom may vary according to factors such as the disease state, age, and weight of the patient, and the ability of the drug to elicit a desired response in the subject. Whether a disease symptom has been alleviated can be assessed by any clinical measurement typically used by physicians or other skilled healthcare providers to assess the severity or progression status of that symptom. The term further includes a postponement of development of the symptoms associated with a disorder and/or a reduction in the severity of the symptoms of such disorder. The terms further include ameliorating existing uncontrolled or unwanted symptoms, preventing additional symptoms, and ameliorating or preventing the underlying causes of such symptoms. Thus, the terms denote that a beneficial result has been conferred on a human or animal subject with a disorder, disease or symptom, or with the potential to develop such a disorder, disease or symptom.

“Treatment” as it applies to a human or veterinary individual, as used herein refers to therapeutic treatment, which encompasses contact of a peptidomimetic macrocycle of the present invention to a human or animal individual who is in need of a treatment with the peptidomimetic macrocycle of the present invention.

### P53 Activating Peptidomimetic Macrocycles

The instant crosslinked peptidomimetic macrocycles have improved properties as compared to known peptidomimetic macrocycles, for example, those disclosed in WO2020112868A. The instant peptidomimetic macrocycles also have improved properties over macrocycles as disclosed in more detail in the following references: A. Burgess, K. M. Chia, S. Haupt, D. Thomas, Y. Haupt and E. Lim. Clinical overview of MDM2/Xtargeted therapies. Front. Oncol., 2016, 6, 1-7; K. Kojima, J. Ishizawa and M. Andreeff. Pharmacological activation of wild-type p53 in the therapy of leukemia. Exp. Hematol., 2016, 44, 791-798; V. Tisato, R. Voltan, A. Gonelli, P. Secchiero and G. Zauli. MDM2/X inhibitors under clinical evaluation: Perspectives for the management of hematological malignancies and pediatric cancer. J. Hematol. Oncol., 2017, 10, 133. In one embodiment, the instant macrocycles have superior binding abilities. In another embodiment, the instant macrocycles have improved physicochemical properties.

The instant crosslinked peptidomimetic macrocycles can have additional MDM2 and/or MDMX(4) binding at the C-terminal tail. In particular, modifications of positions 14 and 17 with lipophilic amino acid residues in the context of a mostly helical C-terminal tail can further boost affinity and/or cellular activity. For example, replacing the alanines at positions 14 and 17 of Macrocycle 3 (SEQ ID NO: 3) with phenylalanines results in Macrocycle 4 (SEQ ID NO: 4). This replacement results in further improved cellular activity in 0% serum relative to Macrocycle 3. Macrocycle 4 shows similar overall helical propensity as compared to Macrocycle 3 in circular dichroism (CD).

In one embodiment, replacing the alanine at position 14 of Macrocycle 3 with a glutamic acid results in Macrocycle 11 (SEQ ID NO: 11) which shows improved cellular activity versus Macrocycle 3. It is believed that the improvement is due to the interaction with MDM2 Arginine 97.

Additional binding at the tail can be engineered with the right set of physicochemical properties in the molecule to improve stability, solubility, and/or cellular activity. In one embodiment, introducing negatively charged alpha amino acid residues to positions 12 and 16 can improve the amphipathicity of the peptides and overall properties. For example, replacing the alanine at position 16 of Macrocycle 4 (SEQ ID NO: 4) with an alpha-methyl glutamic acid results in Macrocycle 9 (SEQ ID NO: 9) which has further improved cellular activity in 10% serum relative to Macrocycle 4. Macrocycle 9 also shows good solubility of 153 uM as compared to close analogs of Macrocycles 5 and 7.

Combining the phenylalanines at positions 14 and 17 with a glutamic acid at position 12 in Macrocycle 12 (SEQ ID NO: 12) results in Macrocycle 13 (SEQ ID NO: 13) which shows further improved binding and cellular activity (both 0% and 10% serum) as compared to Macrocycle 12 and Macrocycle 2. Furthermore, Macrocycle 13 shows improved solubility of 167 uM and excellent overall helical propensity in CD.

Engineering the right set of binding features and physicochemical properties can result in “cleaner” drugs like compounds with improved potency and selectivity. Macrocycle 9 that features an improved amphipathicity profile as compared to Macrocycle 4 shows a selectivity profile in counter screen assay.

Macrocycle 11 that features improved amphipathicity and cellular activity relative to Macrocycle 3 also shows at least 2 fold improvement in p53-dependent HTC116 cell proliferation assay and a cleaner profile in p53-nul Ca Ski counter screen proliferation assay.

Additionally, Macrocycle 13 which shows improved cellular activity profile at 10% serum results in greater than 7 fold improvement in HTC116 cell proliferation assay as compared to Macrocycle 2.

Additional modifications at the staple can result in further improvement in the overall properties. For example, the use of a di-alkyne staple in Macrocycle 13 provides additional benefits in improving potency for cellular proliferation assay.

While only staples between X₁ and X₂ have been described here, additional staples linking L and E within L-T-F-X₁-E (SEQ ID NO: 23) or linking Z₁ and Z₅ within Z₁-Z₂-Z₃-Z₄-Z₅ (as lactams or carbon-based i, i+staples for example) are conceivable.

Thus, the alternative tails disclosed herein provide improved binding and physicochemical properties. Unexpected properties are also obtained in an alkyne staple leading to clean peptides for their on versus off target profile. Combinations of staple type, staple location and C-terminal modifications can lead to unexpected biological specificity and activity. Overall, the peptidomimetic macrocycles disclosed here provide improved biological profiles.

### Pharmaceutical Compositions

Disclosed herein also are pharmaceutical compositions comprising a peptidomimetic macrocycle of the present disclosure. The peptidomimetic macrocycle may be used in combination with any suitable pharmaceutical carrier or excipient. Such pharmaceutical compositions comprise a therapeutically effective amount of one or more peptidomimetic macrocycles, and pharmaceutically acceptable excipient(s) and/or carrier(s). The specific formulation will suit the mode of administration. In particular aspects, the pharmaceutical acceptable carrier may be water or a buffered solution.

Excipients included in the pharmaceutical compositions have different purposes depending, for example on the nature of the drug, and the mode of administration. Examples of generally used excipients include, without limitation: saline, buffered saline, dextrose, water-for-infection, glycerol, ethanol, and combinations thereof, stabilizing agents, solubilizing agents and surfactants, buffers and preservatives, tonicity agents, bulking agents, lubricating agents (such as talc or silica, and fats, such as vegetable stearin, magnesium stearate or stearic acid), emulsifiers, suspending or viscosity agents, inert diluents, fillers (such as cellulose, dibasic calcium phosphate, vegetable fats and oils, lactose, sucrose, glucose, mannitol, sorbitol, calcium carbonate, and magnesium stearate), disintegrating agents (such as crosslinked polyvinyl pyrrolidone, sodium starch glycolate, crosslinked sodium carboxymethyl cellulose), binding agents (such as starches, gelatin, cellulose, methyl cellulose or modified cellulose such as microcrystalline cellulose, hydroxypropyl cellulose, sugars such as sucrose and lactose, or sugar alcohols such as xylitol, sorbitol or maltitol, polyvinylpyrrolidone and polyethylene glycol), wetting agents, antibacterials, chelating agents, coatings (such as a cellulose film coating, synthetic polymers, shellac, corn protein zein or other polysaccharides, and gelatin), preservatives (including vitamin A, vitamin E, vitamin C, retinyl palmitate, and selenium, cysteine, methionine, citric acid and sodium citrate, and synthetic preservatives, including methyl paraben and propyl paraben), sweeteners, perfuming agents, flavoring agents, coloring agents, administration aids, and combinations thereof.

Carriers are compounds and substances that improve and/or prolong the delivery of an active ingredient to a subject in the context of a pharmaceutical composition. Carriers may serve to prolong the in vivo activity of a drug or slow the release of the drug in a subject, using controlled-release technologies. Carriers may also decrease drug metabolism in a subject and/or reduce the toxicity of the drug. Carriers can also be used to target the delivery of the drug to particular cells or tissues in a subject. Common carriers (both hydrophilic and hydrophobic carriers) include fat emulsions, lipids, PEGylated phospholipids, PEGylated liposomes, PEGylated liposomes coated via a PEG spacer with a cyclic RGD peptide c(RGD^{DYK (SEQ ID NO: 24)}), liposomes and lipospheres, microspheres (including those made of biodegradable polymers or albumin), polymer matrices, biocompatible polymers, protein-DNA complexes, protein conjugates, erythrocytes, vesicles, nanoparticles, and side-chains for hydro-carbon stapling. The aforementioned carriers can also be used to increase cell membrane permeability of the peptidomimetic macrocycles of the invention.

Pharmaceutical compositions adapted for oral administration may be presented as discrete units such as capsules or tablets; as powders or granules; as solutions, syrups or suspensions (in aqueous or non-aqueous liquids; or as edible foams or whips; or as emulsions). Suitable excipients for tablets or hard gelatin capsules include lactose, maize starch or derivatives thereof, stearic acid or salts thereof. Suitable excipients for use with soft gelatin capsules include for example vegetable oils, waxes, fats, semi-solid, or liquid polyols etc. For the preparation of solutions and syrups, excipients which may be used include for example water, polyols and sugars. For the preparation of suspensions oils, e.g., vegetable oils, may be used to provide oil-in-water or water in oil suspensions. In certain situations, delayed release preparations may be advantageous and compositions which can deliver the peptidomimetic macrocycles in a delayed or controlled release manner may also be prepared. Prolonged gastric residence brings with it the problem of degradation by the enzymes present in the stomach and so enteric-coated capsules may also be prepared by standard techniques in the art where the active substance for release lower down in the gastro-intestinal tract.

Pharmaceutical compositions adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6):318 (1986).

Pharmaceutical compositions adapted for topical administration may be formulated as ointments, creams, suspensions, lotions, powders, solutions, pastes, gels, sprays, aerosols or oils. When formulated in an ointment, the active ingredient may be employed with either a paraffinic or a water-miscible ointment base. Alternatively, the active ingredient may be formulated in a cream with an oil-in-water cream base or a water-in-oil base. Pharmaceutical compositions adapted for topical administration to the eye include eye drops wherein the active ingredient is dissolved or suspended in a suitable carrier, especially an aqueous solvent. Pharmaceutical compositions adapted for topical administration in the mouth include lozenges, pastilles and mouth washes.

Pharmaceutical compositions adapted for rectal administration may be presented as suppositories or enemas.

Pharmaceutical compositions adapted for nasal administration wherein the carrier is a solid include a coarse powder having a particle size for example in the range 20 to 500 microns which is administered in the manner in which snuff is taken, i.e., by rapid inhalation through the nasal passage from a container of the powder held close up to the nose. Suitable compositions wherein the carrier is a liquid, for administration as a nasal spray or as nasal drops, include aqueous or oil solutions of the active ingredient.

Pharmaceutical compositions adapted for administration by inhalation include fine particle dusts or mists which may be generated by means of various types of metered dose pressurized aerosols, nebulizers or insufflators.

Pharmaceutical compositions adapted for vaginal administration may be presented as pessaries, tampons, creams, gels, pastes, foams or spray formulations.

Pharmaceutical compositions adapted for parenteral administration include aqueous and non-aqueous sterile injection solution which may contain anti-oxidants, buffers, bacteriostats and solutes which render the formulation substantially isotonic with the blood of the intended recipient; and aqueous and non-aqueous sterile suspensions which may include suspending agents and thickening agents. Excipients which may be used for injectable solutions include water-for-injection, alcohols, polyols, glycerin and vegetable oils, for example. The compositions may be presented in unit-dose or multi-dose containers, for example sealed ampoules and vials, and may be stored in a freeze-dried (lyophilized) condition requiring only the addition of the sterile liquid carrier, for example water or saline for injections, immediately prior to use. Extemporaneous injection solutions and suspensions may be prepared from sterile powders, granules and tablets. The pharmaceutical compositions may contain preserving agents, solubilizing agents, stabilizing agents, wetting agents, emulsifiers, sweeteners, colorants, odorants, salts (substances of the present invention may themselves be provided in the form of a pharmaceutically acceptable salt), buffers, coating agents or antioxidants. They may also contain therapeutically active agents in addition to the substance of the present invention.

The pharmaceutical compositions may be administered in a convenient manner such as by the topical, intravenous, intraperitoneal, intramuscular, intratumor, subcutaneous, intranasal or intradermal routes. The pharmaceutical compositions are administered in an amount which is effective for treating and/or prophylaxis of the specific indication. In general, the pharmaceutical compositions are administered in an amount of at least about 0.1 mg/kg to about 100 mg/kg body weight. In most cases, the dosage is from about 10 mg/kg to about 1 mg/kg body weight daily, taking into account the routes of administration, symptoms, etc.

Dosages of the peptidomimetic macrocycles of the present disclosure can vary between wide limits, depending upon the location, source, identity, extent and severity of the cancer, the age and condition of the individual to be treated, etc. A physician will ultimately determine appropriate dosages to be used.

The peptidomimetic macrocycles may also be employed in accordance with the present disclosure by expression of the antagonists in vivo, i.e., via gene therapy. The use of the peptides or compositions in a gene therapy setting is also considered to be a type of “administration” of the peptides for the purposes of the present invention.

Accordingly, the present invention also relates to methods of treating a subject having cancer, comprising administering to the subject a pharmaceutically-effective amount of one or more peptidomimetic macrocycle of the present invention, or a pharmaceutical composition comprising one or more of the antagonists to a subject needing treatment. The term “cancer” is intended to be broadly interpreted and it encompasses all aspects of abnormal cell growth and/or cell division. Examples include: carcinoma, including but not limited to adenocarcinoma, squamous cell carcinoma, adenosquamous carcinoma, anaplastic carcinoma, large cell carcinoma, small cell carcinoma, and cancer of the skin, breast, prostate, bladder, vagina, cervix, uterus, liver, kidney, pancreas, spleen, lung, trachea, bronchi, colon, small intestine, stomach, esophagus, gall bladder; sarcoma, including but not limited to chondrosarcoma, Ewing's sarcoma, malignant hemangioendothelioma, malignant schwannoma, osteosarcoma, soft tissue sarcoma, and cancers of bone, cartilage, fat, muscle, vascular, and hematopoietic tissues; lymphoma and leukemia, including but not limited to mature B cell neoplasms, such as chronic lymphocytic leukemia/small lymphocytic lymphoma, B-cell prolymphocytic leukemia, lymphomas, and plasma cell neoplasms, mature T cell and natural killer (NK) cell neoplasms, such as T cell prolymphocytic leukemia, T cell large granular lymphocytic leukemia, aggressive NK cell leukemia, and adult T cell leukemia/lymphoma, Hodgkin lymphomas, and immunodeficiency-associated lymphoproliferative disorders; germ cell tumors, including but not limited to testicular and ovarian cancer; blastoma, including but not limited to hepatoblastoma, medulloblastoma, nephroblastoma, neuroblastoma, pancreatoblastoma, leuropulmonary blastoma and retinoblastoma. The term also encompasses benign tumors.

In each of the embodiments, the individual or subject receiving treatment is a human or non-human animal, e.g., a non-human primate, bird, horse, cow, goat, sheep, a companion animal, such as a dog, cat or rodent, or another mammal. In some embodiments, the subject is a human.

Also provided is a kit comprising one or more containers filled with one or more of the ingredients of the pharmaceutical compositions of the disclosure, such as a container filled with a pharmaceutical composition comprising a peptidomimetic macrocycle of the present disclosure and a pharmaceutically acceptable carrier or diluent. Associated with such container(s) can be a notice in the form prescribed by a governmental agency regulating the manufacture, use or sale of pharmaceuticals or biological products, which notice reflects approval by the agency of manufacture, use or sale for human administration. In addition, the pharmaceutical compositions may be employed in conjunction with other therapeutic compounds.

### Combination Therapy Comprising Chemotherapy

The peptidomimetic macrocycles disclosed herein may be administered to an individual having a cancer in combination with chemotherapy. The individual may undergo the chemotherapy at the same time the individual is administered the peptidomimetic macrocycle. The individual may undergo chemotherapy after the individual has completed a course of treatment with the peptidomimetic macrocycle. The individual may be administered the peptidomimetic macrocycle after the individual has completed a course of treatment with a chemotherapy agent. The combination therapy of the present invention may also be administered to an individual having recurrent or metastatic cancer with disease progression or relapse cancer and who is undergoing chemotherapy or who has completed chemotherapy.

The chemotherapy may include a chemotherapy agent selected from the group consisting of:
(i) alkylating agents, including but not limited to, bifunctional alkylators, cyclophosphamide, mechlorethamine, chlorambucil, and melphalan;
(ii) monofunctional alkylators, including but not limited to, dacarbazine, nitrosoureas, and temozolomide (oral dacarbazine);
(iii) anthracyclines, including but not limited to, daunorubicin, doxorubicin, epirubicin, idarubicin, mitoxantrone, and valrubicin;
(iv) cytoskeletal disruptors (taxanes), including but not limited to, paclitaxel, docetaxel, abraxane, and taxotere;
(v) epothilones, including but not limited to, ixabepilone, and utidelone;
(vi) histone deacetylase inhibitors, including but not limited to, vorinostat, and romidepsin;
(vii) inhibitors of topoisomerase i, including but not limited to, irinotecan, and topotecan;
(viii) inhibitors of topoisomerase ii, including but not limited to, etoposide, teniposide, and tafluposide;
(ix) kinase inhibitors, including but not limited to, bortezomib, erlotinib, gefitinib, imatinib, vemurafenib, and vismodegib;
(x) nucleotide analogs and precursor analogs, including but not limited to, azacitidine, azathioprine, fluoropyrimidines (e.g., such as capecitabine, carmofur, doxifluridine, fluorouracil, and tegafur) cytarabine, gemcitabine, hydroxyurea, mercaptopurine, methotrexate, and tioguanine (formerly thioguanine);
(xi) peptide antibiotics, including but not limited to, bleomycin and actinomycin; a platinum-based agent, including but not limited to, carboplatin, cisplatin, and oxaliplatin;
(xii) retinoids, including but not limited to, tretinoin, alitretinoin, and bexarotene; and (xiii) vinca alkaloids and derivatives, including but not limited to, vinblastine, vincristine, vindesine, and vinorelbine.

Selecting a dose of the chemotherapy agent for chemotherapy depends on several factors, including the serum or tissue turnover rate of the entity, the level of symptoms, the immunogenicity of the entity, and the accessibility of the target cells, tissue or organ in the individual being treated. The dose of the additional therapeutic agent should be an amount that provides an acceptable level of side effects. Accordingly, the dose amount and dosing frequency of each additional therapeutic agent will depend in part on the particular therapeutic agent, the severity of the cancer being treated, and patient characteristics. Guidance in selecting appropriate doses of antibodies, cytokines, and small molecules are available. See. e.g., Wawrzynczak (1996) Antibody Therapy, Bios Scientific Pub. Ltd, Oxfordshire, UK; Kresina (ed.) (1991) Monoclonal Antibodies. Cytokines and Arthritis, Marcel Dekker, New York, NY; Bach (ed.) (1993) Monoclonal Antibodies and Peptide Therapy in Autoimmune Diseases, Marcel Dekker, New York, NY; Baert et al. (2003) New Engl. J. Med. 348:601-608; Milgrom et al. (1999) New Engl. J. Med. 341:1966-1973; Slamon et al. (2001) New Engl. J. Med. 344:783-792; Beniaminovitz et al. (2000) New Engl. J. Med. 342:613-619; Ghosh et al. (2003) New Engl. J. Med. 348:24-32; Lipsky et al. (2000) New Engl. J. Med. 343:1594-1602; Physicians' Desk Reference 2003 (Physicians' Desk Reference, 57th Ed); Medical Economics Company; ISBN: 1563634457; 57th edition (November 2002). Determination of the appropriate dose regimen may be made by the clinician, e.g., using parameters or factors known or suspected in the art to affect treatment or predicted to affect treatment, and will depend, for example, the individual's clinical history (e.g., previous therapy), the type and stage of the cancer to be treated and biomarkers of response to one or more of the therapeutic agents in the combination therapy.

Contemplated are embodiments of the combination therapy that include a chemotherapy step comprising platinum-containing chemotherapy, pemetrexed and platinum chemotherapy or carboplatin and either paclitaxel or nab-paclitaxel. In particular embodiments, the combination therapy with a chemotherapy step may be used for treating at least NSCLC and HNSCC.

The combination therapy may be used for the treatment any proliferative disease, in particular, treatment of cancer. In particular embodiments, the combination therapy may be used to treat melanoma, non-small cell lung cancer, head and neck cancer, urothelial cancer, breast cancer, gastrointestinal cancer, multiple myeloma, hepatocellular cancer, non-Hodgkin lymphoma, renal cancer, Hodgkin lymphoma, mesothelioma, ovarian cancer, small cell lung cancer, esophageal cancer, anal cancer, biliary tract cancer, colorectal cancer, cervical cancer, thyroid cancer, or salivary cancer.

In another embodiment, the combination therapy may be used to treat pancreatic cancer, bronchus cancer, prostate cancer, pancreatic cancer, stomach cancer, ovarian cancer, urinary bladder cancer, brain or central nervous system cancer, peripheral nervous system cancer, uterine or endometrial cancer, cancer of the oral cavity or pharynx, liver cancer, kidney cancer, testicular cancer, biliary tract cancer, small bowel or appendix cancer, adrenal gland cancer, osteosarcoma, chondrosarcoma, or cancer of hematological tissues.

In particular embodiments, the combination therapy may be used to treat one or more cancers selected from melanoma (metastatic or unresectable), primary mediastinal large B-cell lymphoma (PMBCL), urothelial carcinoma, MSIHC, gastric cancer, cervical cancer, hepatocellular carcinoma (HCC), Merkel cell carcinoma (MCC), renal cell carcinoma (including advanced), and cutaneous squamous carcinoma.

### Additional Combination Therapies

The peptidomimetic macrocycles disclosed herein may be used in combination with other therapies. For example, the combination therapy may include a composition comprising a peptidomimetic macrocycle co-formulated with, and/or co-administered with, one or more additional therapeutic agents, e.g., hormone treatment, vaccines, and/or other immunotherapies. In other embodiments, the peptidomimetic macrocycle is administered in combination with other therapeutic treatment modalities, including surgery, radiation, cryosurgery, and/or thermotherapy. Such combination therapies may advantageously utilize lower dosages of the administered therapeutic agents, thus avoiding possible toxicities or complications associated with the various monotherapies.

By “in combination with,” it is not intended to imply that the therapy or the therapeutic agents must be administered at the same time and/or formulated for delivery together, although these methods of delivery are within the scope described herein. The peptidomimetic macrocycle may be administered concurrently with, prior to, or subsequent to, one or more other additional therapies or therapeutic agents. The peptidomimetic macrocycle and the other agent or therapeutic protocol may be administered in any order. In general, each agent will be administered at a dose and/or on a time schedule determined for that agent. In will further be appreciated that the additional therapeutic agent utilized in this combination may be administered together in a single composition or administered separately in different compositions. In general, it is expected that additional therapeutic agents utilized in combination be utilized at levels that do not exceed the levels at which they are utilized individually. In some embodiments, the levels utilized in combination will be lower than those utilized individually.

In certain embodiments, a peptidomimetic macrocycle described herein is administered in combination with one or more check point inhibitors or antagonists of programmed death receptor 1 (PD-1) or its ligand PD-L1 and/or PD-L2. The inhibitor or antagonist may be an antibody, an antigen binding fragment, an immunoadhesin, a fusion protein, or oligopeptide. In some embodiments, the anti-PD-1 antibody is chosen from nivolumab (OPDIVO, Bristol Myers Squibb, New York, New York), pembrolizumab (KEYTRUDA, Merck Sharp & Dohme Corp, Kenilworth, NJ USA), cetiplimab (Regeneron, Tarrytown, NY) or pidilizumab (CT-011). In some embodiments, the PD-1 inhibitor is an immunoadhesin (e.g., an immunoadhesin comprising an extracellular or PD-1 binding portion of PD-L1 or PD-L2 fused to a constant region (e.g., an Fc region of an immunoglobulin sequence)). In some embodiments, the PD-1 inhibitor is AMP-224. In some embodiments, the PD-L1 inhibitor is anti-PD-L1 antibody such durvalumab (IMFINZI, AstraZeneca, Wilmington, DE), atezolizumab (TECENTRIQ, Roche, Zurich, CH), or avelumab (BAVENCIO, EMD Serono, Billerica, MA). In some embodiments, the anti-PD-L1 binding antagonist is chosen from YW243.55.S70, MPDL3280A, MEDI-4736, MSB-0010718C, or MDX-1105.

The following examples are intended to promote a further understanding of the present invention.

### General Methods

Available crystal structure of ATSP-7041 co-crystalized with MDM4 were used to model stapled peptides with C-terminal extension. All these models were subjected to MD (Molecular Dynamics) simulations for further refinement. MD simulations were carried out on the free peptide and peptide-MDM2 complexes. The Xleap module of AMBERi6 was used to prepare the system for the MD simulations. Hydrogen atoms were added and the N-terminus, C-terminus of the peptide was capped with residues ACE (acetyl) and NHE (primary amide). The parameters for the staple linkers were taken from a previous study (Tan, Y. S. et al. Benzene Probes in Molecular Dynamics Simulations Reveal Novel Binding Sites for Ligand Design. J Phys Chem Lett 7, 3452-3457). All the simulation systems were neutralized with appropriate numbers of counter ions. The neutralized system was solvated in an octahedral box with TIP3P water molecules, leaving at least 10 Å between the solute atoms and the borders of the box. MD simulations were carried out with AMBER 16 package in combination with the ff14SB force field. All MD simulations were carried out in explicit solvent at 300K. During all the simulations the long-range electrostatic interactions were treated with the particle mesh Ewald method using a real space cut off distance of 9 Å. The settle algorithm was used to constrain bond vibrations involving hydrogen atoms, which allowed a time step of 2 fs during the simulations.

Solvent molecules and counter ions were initially relaxed using energy minimization with restraints on the protein and peptide atoms. This was followed by unrestrained energy minimization to remove any steric clashes. Subsequently the system was gradually heated from 0 to 300° K using MD simulations with positional restraints (force constant: 50 kcal mol⁻¹ Å⁻²) on protein and peptides over a period of 0.25 ns allowing water molecules and ions to move freely followed by gradual removal of the positional restraints and a 2 ns unrestrained equilibration at 300° K. The resulting systems were used as starting structures for the respective production phases of the MD simulations. For each case, three independent (using different initial random velocities) MD simulations were carried out starting from the well equilibrated structures. Each MD simulation was carried out for 250 ns and conformations were recorded every 4 ps.

To enhance the conformational sampling, each of these peptides were subjected to Biasing Potential Replica Exchange Molecular Dynamics (BP-REMD) simulations. The BP-REMD technique is a type of Hamiltonian-REMD methods which includes a biasing potential that promote dihedral transitions along the replicas (Kannan S, Zacharias M (2007) Enhanced sampling of peptide and protein conformations using replica exchange simulations with a peptide backbone biasing-potential. Proteins. 66: 697-706; Ostermeir K, Zacharias M. Hamiltonian replica-exchange simulations with adaptive biasing of peptide backbone and side chain dihedral angles. J. Comput. Chem., 35 (2014)). For each system, BP-REMD was carried with eight replicas including a reference replica without any bias. BP-REMD was carried out for 50 ns with exchange between the neighbouring replicas were attempted for every 2 ps and accepted or rejected according to the metropolis criteria. Conformations sampled at the reference replica (no bias) was used for further analysis. Simulation trajectories were visualized using VMD and figures were generated using Pymol.

### Binding Energy Calculations and Energy Decomposition Analysis:

Molecular Mechanics Poisson Boltzmann Surface Area (MMPBSA) methods were used for the calculation of binding free energies between the peptides and their partner proteins 250 conformations extracted from the last 50 ns of the simulations were used for the binding energy calculations. Entropy calculations are computationally intensive and do not converge easily and hence are ignored. The effective binding energies were decomposed into contributions of individual residues using the MMGBSA energy decomposition scheme. The MMGBSA calculations were carried out in the same way as in the MMPBSA calculations. The polar contribution to the solvation free energy was determined by applying the generalized bom (GB) method (igb=2), using mbondi2 radii. The non-polar contributions were estimated using the ICOSA method by a solvent accessible surface area (SASA) dependent term using a surface tension proportionally constant of 0.0072 kcal/mol A2. The contribution of peptide residues was additionally explored by carrying out in-silico alanine scanning in which each of the peptide residue is mutated to D-alanine in each conformation of the MD simulation and the change with respect to the binding energy of the wild type peptide is calculated using MMPBSA.

### Peptide Synthesis

Peptides were synthesized using RINK Resin and Fmoc-protected amino acids, coupled sequentially with diisopropylcarbodiimide/1-hydroxybenzotriazole (DIC/HOBT) activating agents. Double coupling reactions were performed on the first amino acid and also at the stapling positions. At these latter positions, the activating reagents were switched to N,N-diisopropylethylamine/hexafluorophosphate azabenzotriazole tetramethyl uronium (DIEA/HATU) for better coupling efficiencies.

Ring closing metathesis reactions were performed by first washing the resin 3 times with dichloromethane (DCM), followed by the addition of the 11 generation Grubbs Catalyst (35 mg dissolved into 5 mL DCM) and allowed to react for 2 hours (all steps with Grubbs Catalyst were performed in the dark). The ring-closing metathesis (RCM) reaction was repeated to ensure a complete reaction. After the RCM was complete, a test cleavage was performed to ensure adequate yield. Peptides were cleaved and then purified as a mixture of cis-trans isomers by Reversed Phase-High Performance Liquid Chromatography (RP-HPLC). Glaser alkyne cross coupling reactions were performed on resin by first washing the resin three times with DCM, followed by the addition of tetrahydrofuran (THF), diisopropylethylamine (DIPEA), Pd(PPh₃)₂Cl₂ then CuI, ultrasonication then heating at 30° C. for 16 h. The mixture was filtered and washed with dimethylformamide (DMF).

### MDM2 Protein Production

For use in the peptide binding assay, a human MDM2 1-125 sequence was cloned into a pNIC-GST vector. The TV cleavage site was changed from ENLYFQS (SEQ ID NO: 25) to ENLYFQG (SEQ ID NO: 26) to give a fusion protein with the following sequence (SEQ ID NO 14):

MSDKIIHSPILGYWKIKGLVQPTRLLLEYLEEKYEEHLYERDEGDK
WRNKKFELGLEFPNLPYYIDGDVKLTQSMAIIRYIADKHNMLGGC
PKERAEISMLEGAVLDIRYGVSRIAYSKDFETLKVDFLSKLPEML
KMFEDRLCHKTYLNGDHVTHPDFMLYDALDVVLYMDPMCLDAFPK
LVCFKKRIEAIPQIDKYLKSSKYIAWPLQGWQATFGGGDHPPKLE
VLFQGHMHHHHHHSSGVDLGTENLYFQGMCNTNMSVPTDGAVTTS
QIPASEQETLVRPKPLLLKLLKSVGAQKDTYTMKEVLFYLGQYIM
TKRLYDEKQQHIVYCSNDLLGDLFGVPSFSVKEHRKIYTMIYRNL
VVVNQQESSDSGTSVSEN

The corresponding plasmid was transformed into BL21 (DE3) Rosetta T1R E. coli cells and grown under kanamycin selection. Bottles of 750 mL Terrific Broth (TB) supplemented with appropriate antibiotics and 100 μL of antifoam 204 (Sigma-Aldrich) were inoculated with 20 mL seed cultures grown overnight. The cultures were incubated at 37° C. in the LEX system (Harbinger Biotech) with aeration and agitation through the bubbling of filtered air through the cultures. LEX system temperature was reduced to 18° C. when culture OD600 reached 2, and the cultures were induced after 60 minutes with 0.5 mM IPTG. Protein expression was allowed to continue overnight. Cells were harvested by centrifugation at 4000 g, at 15° C. for 10 min. The supernatants were discarded and the cell pellets were resuspended in lysis buffer (1.5 mL per gram of cell pellet). The cell suspensions were stored at −80° C. before purification work.

The re-suspended cell pellet suspensions were thawed and sonicated (Sonics Vibra-cell) at 70% amplitude, 3 s on/off for 3 minutes, on ice. The lysate was clarified by centrifugation at 47000 g, 4° C. for 25 minutes. The supernatants were filtered through 1.2 um syringe filters and loaded onto AKTA Xpress system (GE Healthcare). The purification regime is briefly described as follows.

The lysates were loaded on to 1 mL Ni-NTA Superflow column (Qiagen) that had been equilibrated with 10 column volumes of wash 1 buffer. Overall buffer conditions were as follows: Immobilized metal affinity chromatography (IMAC) wash 1 buffer: 20 mM 4-(2-hydroxyethyl)-1-piperazineethanesulfonic acid (HEPES), 500 mM NaCl, 10 mM Imidazole, 10% (v/v) glycerol, 0.5 mM tris(2-carboxyethyl)phosphine (TCEP), pH 7.5; IMAC wash 2 buffer: 20 mM HEPES, 500 mM NaCl, 25 mM Imidazole, 10% (v/v) glycerol, 0.5 mM TCEP, pH 7.5; IMAC Elution buffer: 20 mM HEPES, 500 mM NaCl, 500 mM Imidazole, 10% (v/v) glycerol, 0.5 mM TCEP, pH 7.5.

The sample was loaded until air was detected by air sensor, 0.8 mL/minutes. The column was then washed with wash 1 buffer for 20 column volumes followed by 20 column volumes of wash 2 buffer. The protein was eluted with 5 column volumes of elution buffer. The eluted proteins were collected and stored in sample loops on the system and then injected into Gel Filtration (GF) columns. Elution peaks were collected in 2 mL fractions and analysed on SDS-PAGE gels. The entire purification was performed at 4° C. Relevant peaks were pooled, TCEP was added to a total concentration of 2 mM. The protein sample was concentrated in Vivaspin 20 filter concentrators (VivaScience) at 15° C. to approximately 15 mg/mL (<18 kDa—5K MWCO, 19-49 kDa—10K MWCO, >50 kDa—30K MWCO). The final protein concentration was assessed by measuring absorbance at 280 nm on Nanodrop ND-1000 (Nano-Drop Technologies). The final protein purity was assessed on SDS-PAGE gel. The final protein batch was then aliquoted into smaller fractions, frozen in liquid nitrogen and stored at −80° C.

### MDM4 Protein Production

MDM4 protein was cloned into pNIC-GST vector and expressed in LEX system (Harbinger Biotech) at Protein Production Platform (PPP) at NTU (Nanyang Technological University) School of Biological Sciences. Using glycerol stocks, inoculation cultures were started in 20 mL Terrific Broth with 8 g/L glycerol supplemented with Kanamycin. The cultures were incubated at 37° C., 200 rpm overnight. The following morning, bottles of 750 mL Terrific Broth with 8 g/L glycerol supplemented with Kanamycin and 100 μL of antifoam 204 (Sigma-Aldrich) were inoculated with the cultures.

The cultures were incubated at 37° C. in the LEX system with aeration and agitation through the bubbling of filtered air through the cultures. When the OD600 reached ˜2, the temperature was reduced to 18° C. and the cultures were induced after 30 to 60 minutes with 0.5 mM IPTG. Protein expression was allowed to continue overnight. The following morning, cells were harvested by centrifugation at 4200 rpm at 15° C. for 10 minutes. The supernatants were discarded and the cells were re-suspended in lysis buffer (100 mM HEPES, 500 mM NaCl, 10 mM Imidazole, 10% glycerol, 0.5 mM TCEP, pH 8.0 with Benzonase (4 uL per 750 mL cultivation) and 250 U/μL Merck Protease Inhibitor Cocktail Set III, EDTA free (1000× dilution in lysis buffer) from Calbiochem) at 200 rpm, 4° C. for approximately 30 min and stored at −80° C.

The re-suspended cell pellet suspensions were thawed and sonicated (Sonics Vibra-cell) at 70% amplitude, 3 s on/off for 3 minutes, on ice. The lysate was clarified by centrifugation at 47000 g, 4° C. for 25 minutes. The supernatants were filtered through 1.2 μm syringe filters and loaded onto AKTA Xpress system (GE Healthcare) with a 1 mL Ni-NTA Superflow (Qiagen) IMAC column. The column was washed with 20 column volume (CV) of wash buffer 1 (20 mM HEPES, 500 mM NaCl, 10 mM Imidazole, 10% (v/v) glycerol, 0.5 mM TCEP, pH 7.5) and 20 CV of wash buffer 2 (20 mM HEPES, 500 mM NaCl, 25 mM Imidazole, 10% (v/v) glycerol, 0.5 mM TCEP, pH 7.5) or until a stable baseline for 3 min and delta base 5mAU (0.8 mL/min) was obtained respectively.

MDM4 protein was eluted with elution buffer (20 mM HEPES, 500 mM NaCl, 500 mM Imidazole, 10% (v/v) glycerol, 0.5 mM TCEP, pH 7.5) and eluted peaks (start collection: >50mAU, slope>200mAU/minutes, stop collection: <50mAU, stable plateau of 0.5 min, delta plateau 5mAU) were collected and stored in sample loops on the system and then injected into equilibrated Gel Filtration (GF) column (HiLoad 16/60 Superdex 200 prep grade (GE Healthcare)) and eluted with 20 mM HEPES, 300 mM NaCl, 10% (v/v) glycerol, 0.5 mM TCEP, pH 7.5 at a flow rate of 1.2 mL/minutes. Elution peaks (start collection: >20 mAU, slope>10 mAU/min, stop collection: <20mAU, slope>10 mAU/minutes, minimum peak width 0.5 min) were collected in 2 mL fractions. The entire purification was performed at 4° C. Relevant peaks were pooled and TCEP was added to a final concentration of 2 mM. The protein sample was concentrated in Vivaspin 20 filter concentrators (VivaScience) at 15° C. to approximately 15 mg/mL. The final protein concentration was assessed by measuring absorbance at 280 nm on Nanodrop ND-1000 (Nano-Drop Technologies). The final protein purity was assessed by SDS-PAGE and purified MDM4 protein was frozen in liquid nitrogen and stored at −80° C.

### Circular Dichroism (CD)

Prior to the experiment, 5 μl of 10 mM stock peptide was mixed with 45 μl of 100% methanol and dried for 2 hours in the SpeedVac concentrator (Thermo Scientific). The dried peptide was reconstituted in a buffer containing 1 mM HEPES pH 7.4 and 5% methanol to a concentration of 1 mM. The peptide sample was placed in a quartz cuvette with a path length of 0.2 cm and a CD spectrum was recorded from 300 to 190 nm at 25° C. using the Chirascan-plus qCD machine (Applied Photophysics). The actual concentration of the peptide was determined by the absorbance of the peptide at 280 nM. An estimate of the secondary structure components of the peptide was carried out by converting the CD spectrum to mean residue ellipticity before deconvoluting using the CDNN software (distributed by Applied Photophysics). All experiments were done in duplicate.

### Isothermal Titration Calorimetry (ITC)

All experiments were performed in duplicates using the MicroCal PEAQ-ITC Automated system. One hundred to two hundred μM of peptide was titrated into 20 μM of purified recombinant human MDM2 protein (amino acids 1-125; SEQ ID NO 14), over 40 injections of 1 μL each. For peptides that are insoluble at high concentrations, reverse ITC was carried out by titrating 200 μM of MDM2 protein into 20 μM of peptide. All proteins and peptides were dialyzed overnight in a buffer containing 1× phosphate-buffered saline (PBS) pH 7.2, 3% DMSO, and 0.001% Tween-20. Data analysis was carried out using the MicroCal PEAQ-ITC Analysis Software.

### MDM2 Binding Assay

Purified MDM2 (1-125; SEQ ID NO 14) protein was titrated against a 50 nM carboxyfluorescein (FAM)-labeled 12/1 peptide (FAM-RFMDYWEGL-NH₂; SEQ ID NO 15). Dissociation constants for titration of MDM2 against FAM-labeled 12/1 peptide were determined by fitting the experimental data to a 1:1 binding model equation shown below.

$$r = {r}_{o} + ( {r}_{b} - {r}_{o} ) \times \frac{( {K}_{d} + {[ L ]}_{t} + {[ P ]}_{t} ) - \sqrt{{{K}_{d} + {[ L ]}_{t} + {[ P ]}_{t} )}^{2} - {{4 [ L ]}_{t} [ P ]}_{t}}}{{2 [ L ]}_{t}} \qquad Equation 1$$

[P] is the protein concentration (MDM2), [L] is the labeled peptide concentration, r is the anisotropy measured, r₀ is the anisotropy of the free peptide, r_{b} is the anisotropy of the MDM2-FAM-labeled peptide complex, K_{d} is the dissociation constant, [L]t is the total FAM labeled peptide concentration, and [P]ₜ is the total MDM2 concentration. The determined apparent K_{d} value of FAM-labeled 12/1 peptide (13.0 nM) was used to determine the apparent K_{d} values of the respective competing ligands in subsequent competition assays in fluorescence anisotropy experiments. Titrations were carried out with the concentration of MDM2 held constant at 250 nM and the labeled peptide at 50 nM. The competing molecules were then titrated against the complex of the FAM-labeled peptide and protein. Apparent K_{d} values were determined by fitting the experimental data to the equations shown below:

$$\begin{matrix}r = {r}_{o} + ( {r}_{b} + {r}_{o} ) \times \frac{2 \sqrt{( {d}^{2} - 3 e )} cos ( θ / 3 ) - 9}{3 {K}_{d 1} + 2 \sqrt{( {d}^{2} - 3 e )} cos ( θ / 3 ) - d} \\ d = {K}_{d 1} + {K}_{d 2} + {[ L ]}_{st} + {[ L ]}_{t} - {[ P ]}_{t} \\ e = ( {[ L ]}_{t} - {[ P ]}_{t} ) {K}_{d 1} + ( {[ L ]}_{st} - {[ P ]}_{t} ) {K}_{d 2} + {K}_{d 1} {K}_{d 2} \\ f = - {K}_{d 1} {{K}_{d 2} [ P ]}_{t} \\ θ = ar cos [ \frac{- 2 {d}^{3} + 9 de - 27 f}{2 \sqrt{{( {d}^{2} - 3 e )}^{3}}} ]\end{matrix}$$

[L]st and [L]t denote labeled ligand and total unlabeled ligand input concentrations, respectively. K_{d2} is the dissociation constant of the interaction between the unlabeled ligand and the protein. In all competition experiments, it is assumed that [P]ₜ>[L]ₛₜ, otherwise considerable amounts of free labeled ligand would always be present and would interfere with measurements. K_{d1} is the apparent K_{d} for the labeled peptide used and has been experimentally determined as described in the previous paragraph. The FAM-labeled peptide was dissolved in dimethyl sulfoxide (DMSO) at 1 mM and diluted into experimental buffer. Readings were carried out with an Envision Multilabel Reader (PerkinElmer). Experiments were carried out in Phosphate Buffered Saline (PBS) (2.7 mM KCl, 137 mM NaCl, 10 mM Na₂HPO₄ and 2 mM KH₂PO₄ (pH 7.4)) and 0.1% Tween 20 buffer. All titrations were carried out in triplicate. Curve-fitting was carried out using Prism 4.0 (GraphPad). To validate the fitting of a 1:1 binding model, we carefully ensured that the anisotropy value at the beginning of the direct titrations between MDM2 and the FAM-labeled peptide did not differ significantly from the anisotropy value observed for the free fluorescently labeled peptide. Negative control titrations of the ligands under investigation were also carried out with the fluorescently labeled peptide (in the absence of MDM2) to ensure no interactions were occurring between the ligands and the FAM-labeled peptide. In addition, we ensured that the final baseline in the competitive titrations did not fall below the anisotropy value for the free FAM-labeled peptide, which would otherwise indicate an unintended interaction between the ligand and the FAM-labeled peptide to be displaced from the MDM2 binding site.

### p53 Beta-Lactamase Reporter Gene Cellular Functional Assay

HCT116 cells were stably transfected with a p53 responsive β-lactamase reporter, and were seeded into a 384-well plate at a density of 8,000 cells per well. Cells were maintained in McCoy's 5A Medium with 10% fetal bovine serum (Stain Buffer or FBS), Blasticidin and Penicillin/Streptomycin. The cells were incubated overnight and followed by removal of cell growth media and replaced with Opti-MEM either containing 0% FBS or 10% FBS. Peptides were then dispensed to each well using a liquid handler, ECHO 555 and incubated for 4/16 hours. Final working concentration of dimethyl sulfoxide (DMSO) was 0.5%. β-Lactamase activity was detected using the ToxBLAzer Dual Screen (Invitrogen) as per manufacturer's instructions. Measurements were done using Envision multiplate reader (Perkin-Elmer). Maximum p53 activity was defined as the amount of β-lactamase activity induced by 50 μM azide-ATSP-7041 (stapled p53 peptide; Aileron Therapeutics, Inc.). This was determined as the highest amount of p53 activity induced by azide-ATSP-7041 by titration on HCT116 cells.

### Lactate Dehydrogenase Release Assay (LDH)

HCT116 cells were seeded into a 384-well plate at a density of 8000 cells per well. Cells were maintained in McCoy's 5A Medium with 10% fetal bovine serum (FBS), Blasticidin and Penicillin/Streptomycin. The cells were incubated overnight followed by the removal of cell media and the addition of Opti-MEM Medium without FBS. Cells were then treated with peptides for 4/16 hours in Opti-MEM either in 10% FBS or serum free. Final concentration of DMSO was 0.5%. Lactate dehydrogenase release was detected using CytoTox-ONE Homogenous Membrane Integrity Assay Kit (Promega) as per manufacturer's instructions. Measurements were carried out using Tecan plate reader. Maximum LDH release was defined as the amount of LDH released induced by the lytic peptide (iDNA79) and used to normalize the results.

### Tetracycline Beta-Lactamase Reporter Gene Assay (Counter Screen)

Based on Jump-In™ T-REx™ CHO-K1 BLA cells and contain a stably integrated β-lactamase under the control of an inducible CMV promoter. Cells were seeded into a 384-well plate a density of 4000 cells per well. Cells were maintained in Dulbecco's Minimal Eagle Medium (DMEM) with 10% fetal bovine serum (FBS), Blasticidin and Penicillin/Streptomycin. The cells were incubated for 24 hours, followed by cell media removal and replacement with Opti-MEM either containing 10% FBS or 0% FBS. Peptides were then dispensed to each well using a liquid handler, ECHO 555 and incubated for 4/16 hours. Final working concentration of DMSO was 0.5%. β-Lactamase activity was detected using the ToxBLAzer Dual Screen (Invitrogen) as per manufacturer's instructions. Measurements were carried out using Envision multiplate reader (Perkin Elmer). Counter Screen activity was defined as the amount of 0-lactamase activity induced by tetracycline.

### HCT-116 Western Blot Analysis

Preparation of Compound Stock and Working Solutions: 10 mM or 1 mM stock solutions of compounds were prepared in 100% DMSO. Each compound was then serially diluted in 100% DMSO and further diluted 10-fold into HPLC grade sterile water to prepare 10× working solutions in 10% DMSO/water of each compound. Depending on the required volume used in the relevant assay, compounds were added to yield final concentrations as indicated in the relevant figure with a residual DMSO concentration of 1% v/v.

HCT116 cells (Thermo Fisher Scientific) were cultured in DMEM cell media, which was supplemented with 10% fetal calf serum (FBS) and penicillin/streptomycin. All cell lines were maintained in a 37° C. humidified incubator with 5% CO₂ atmosphere. HCT116 cells were seeded into 96 well plates at a cell density of 60,000 cells per well and incubated overnight. Cells were also maintained in DMEM cell media with 10% fetal bovine serum (FBS) and penicillin/streptomycin. Cell media was then removed and replaced with cell media containing the various compounds/vehicle controls at the concentrations indicated in DMEM cell media with 2% FCS. After the stated incubation time (4 or 24 hours) cells were rinsed with PBS and then harvested in 100 μl of 1× NuPAGE LDS sample buffer supplied by Invitrogen (NP0008). Samples were then sonicated, heated to 90° C. for 5 minutes, sonicated twice for 10 seconds and centrifuged at 13,000 rpm for 5 minutes. Protein concentrations were measured by BCA assay (Pierce). Samples were resolved on Tris-Glycine 4-20% gradient gels (BIORAD) according to the manufacturer's protocol. Western transfer was performed with an Immuno-blot PVDF membrane (Bio-Rad) using a Trans-Blot Turbo system (BIORAD). Western blot staining was then performed using antibodies against actin (AC-15, Sigma) as a loading control, p21 (118 mouse monoclonal), MDM2 (2A9 mouse monoclonal antibody) and p53 (DO-1 mouse monoclonal antibody).

### Example 1

Preparation of Azido ATSP-7041 Macrocycles with C-Terminal Extensions

WO 2020112868A describes a series of ATSP-7041 peptide analogs appended with a number of alanine residues (herein described as polyalanine tail) at the C-terminal tail. Previously, an azido-ATSP-7041 peptide with an S12A substitution was made, resulting in a triple Ala sequence (3xA) C-terminal to the S5 stapling position. This peptide (Reference Macrocycle 1) had activities of 339 nM (0% serum at 4 hours) and 847 nM (10% serum at 16 hours) in the p53 cellular assay (Table 3). When the C-terminal was extended by three more Ala residues to give 6xA tail Macrocycle 2, the cellular activity was improved by ˜3.5 fold in both 0% serum at 4 hours, and 10% serum at 16 hours. Macrocycle 3 was also made containing a 5xA-dA tail and it behaved similarly to Macrocycle 2 in cellular assay, and the solubility was improved versus Reference Macrocycle 1 (Tables 3 and 4).

### Example 2

### Modification of a C-Terminal Tail Improves Cellular Uptake in 0% Serum

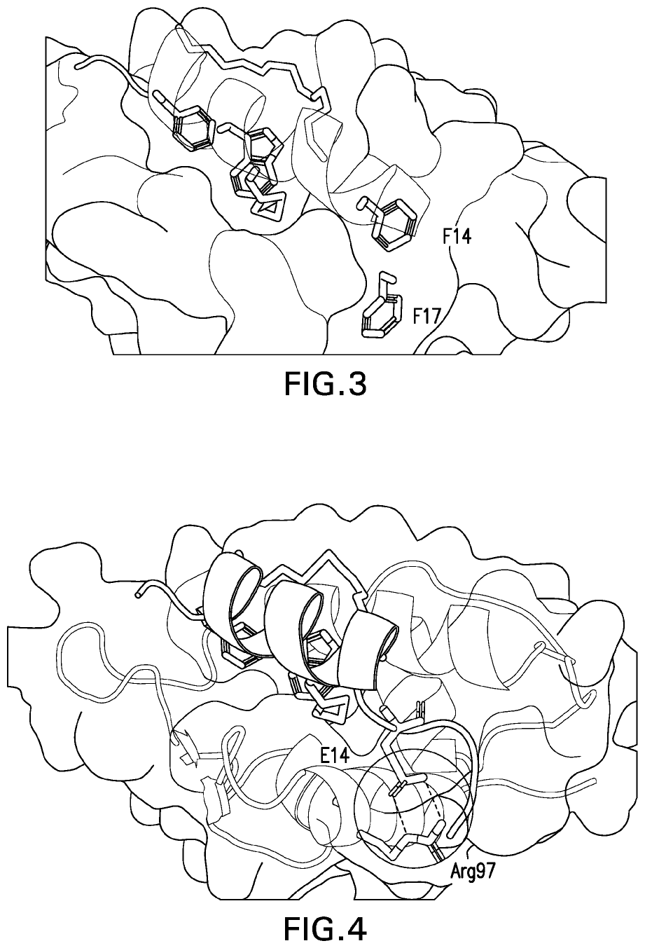

The C-terminal tail provides opportunities to further enhance cell potency by optimizing the C-terminal tail sequence. Examination of the helical wheel (FIG. 2) revealed that placement of apolar residues at positions 14 and/or 17 might enhance MDM2 binding. Molecular modeling suggested that placing one or more phenylalanine in these positions might improve MDM2 binding and other properties (FIG. 3).

A series of peptides (Macrocycles 4 to 8) were made where one or more Phe residues were placed into positions 14 and/or 17, including using alpha methylation (see Table 1 for amino acid sequences). The placement of apolar residues at these positions further enhanced MDM2 binding. Such Macrocycles also showed enhanced or comparable cellular activity in the 0% FBS (serum), 4-hour assay as compared to Macrocycles 2 and 3 (e.g., 36 nM for Macrocycle 4 versus 143 nM for Macrocycle 2).

### Example 3

### Placement of a Charged Residue at Position 16 Results in Improved Properties

In general, Macrocycles 4 to 8 showed increased hydrophobicity as shown by their higher A log P98 and/or HPLC Log D (Tables 3 and 4). It is now found that the placement of a charged residue at position 16 further improves solubility and cell potency while maintaining MDM2 binding. For example, Macrocycle 9 which has an alpha methyl glutamic acid at this position was made which showed improved cell potency at 16 hours, 10% condition as compared to Macrocycle 4 (Table 3). It also showed improved solubility of 153 uM as compared to Macrocycles 5 and 7 which are close analogs of Macrocycle 4. Macrocycle 9 also showed improved cell potency at 16 hours, 10% condition as compared to Macrocycle 2. It has thus been shown that the cellular activity and solubility of crosslinked peptidomimetic macrocycles can be improved by modifications at the C-terminal tail as disclosed herein.

### Example 4

### Introduction of Amphiphatic Residues at the Tail Improves Physiochemical Properties

It is found that introduction of amphipathicity at the C-terminal tail can result in clean “drug” like compounds with improved solubility and other properties. For example, a glutamic acid or gamma-carboxyglutamic acid was discretely introduced at the tail of Macrocycle2 or 3 resulting in Macrocycles 10 and 11. Macrocycle 10 with glutamic acid at position 14 showed improved cellular activity in 10% serum at 16 hours as compared to Macrocycle 3. Molecular modeling suggests that a glutamic acid at position 14 might engage with an MDM2 arginine, Arg97 (FIG. 4). Macrocycle 11 with a carboxylic glutamic acid (Gla) at position 12 showed improved binding versus Macrocycle 2, and a cellular activity with ˜2 fold at 527 nM in 10% serum, 16 hours (Table 3). Both Macrocycles 10 and 11 show excellent solubility (Table 4).

Macrocycles 10 and 11 were also profiled in cellular proliferation assays in conjunction with Macrocycle 2 (Table 5). Macrocycle 10 showed a ˜2 fold improvement in cellular efficacy in HTC116 p53 positive control cell lines at 187 nM, as compared to Macrocycle 3 at 420 nM. Macrocycle 11 showed cellular activity within ˜2 fold of Macrocycle 3.

In addition, both Macrocycles 10 and 11 were clean with a lack of liability in p53 null negative control cell line (>50 uM EC₅₀ and −18% inhibition at maximum concentration), as compared to Macrocycle 2 that showed some residual activity at the highest assay concentration (29% inhibition at 50 uM). Macrocycles 10 and 11 thus highlight the applicability of various modifications at the C-terminal tail to provide compounds with improved cellular activity in cellular assay and/or Counter Screen assays.

### Example 5

### Using a Bis-Alkyne in Lieu of the Alkene Staple Improved Potency in Cellular Proliferation Assay

Macrocycle 12 that contains bis-alkyne replacement and the polyalanine C-terminal tail was made. This Macrocycle showed comparable cellular activity (within 2 fold) relative to Macrocycle 3 in both 0% and 10% serum (Table 3). It also showed similar solubility (Table 4). Most significantly, it improved cellular efficacy in cell proliferation HTC116 p53 positive control cell lines by >3 fold to 124 nM, as compared to Macrocycle 3 at 420 nM (Table 4). It was also clean with a total lack of liability in p53 null negative control cell line (>50 uM EC₅₀ and 3% inhibition at maximum concentration).

The di-alkyne motif which was introduced to the C-terminal tail of Macrocycle 4 showed excellent cellular activity in 0% serum at 4 hours. To balance hydrophobicity and improve amphiphaticity and cellular activity profile in Counter Screen, a glutamic acid was also introduced at position 12. The resulting Macrocycle 13 showed comparable potency to Macrocycle 4 in 0% serum at 4 hours (51 vs 36 nM) and significantly improved potency in 10% serum at 16 hours as compared to Macrocycles 4 and 2 (153 nM vs 237 and 749 nM). Macrocycle 13 also showed excellent solubility at 167 uM. Most importantly, it improved cellular efficacy in cell proliferation HTC116 p53 positive control cell lines, by >15 fold as compared to Macrocycle 4 (73 vs 1215 nM, respectively) and by >7 fold as compared to Macrocycle 2 (73 vs 570 nM, respectively) (Table 5).

Thus, macrocycles disclosed herein demonstrate the applicability of various modifications to provide potent cellularly active and clean compounds.

TABLE 3
Binding and cellular activity of stapled ATSP-7041 C-terminal
peptides determined through various biochemical methods

| | | p53 | p53 | LDH | LDH | Counter | Counter |
|---|---|---|---|---|---|---|---|
| | | activity | activity | release | release | screen | screen |
| | FP Kd | EC₅₀ (uM) | EC₅₀ (uM) | EC₅₀ (uM) | EC₅₀ (uM) | EC₅₀ (uM) | EC₅₀ (uM) |
| SEQ | MDM2 | (0% FBS, | (10% FBS, | (0% FBS, | (10% FBS, | (0% FBS, | (10% FBS, |
| ID NO | (nM) | 4 h) | 16 h) | 4 h) | 16 h) | 4 h) | 16 h) |
| 1 | 36 | 339 | 847 | >50 | nd | >50 | nd |
| 2 | 9.4 | 143 | 237 | >50 | >50 | >50 | >50 |
| 3 | 31 | 432 | 292 | >50 | >50 | >50 | >50 |
| 4 | 27 | 36 | 749 | >50 | >50 | 34.5 | >50 |
| 5 | 56 | 110 | 2133 | >50 | >50 | >50 | >50 |
| 6 | 2.1 | 167.3 | 2663 | >50 | >50 | >50 | >50 |
| 7 | 4.2 | 208 | 686 | >50 | >50 | >50 | >50 |
| 8 | 7.4 | 228 | 751 | 16.7 | >50 | >50 | >50 |
| 9 | 4.8 | 198 | 206 | >50 | >50 | >50 | >50 |
| 10 | 3.1 | 959 | 194 | >50 | >50 | >50 | >50 |
| 11 | 0.32 | 785 | 527 | >50 | >50 | >50 | >50 |
| 12 | 5.0 | 246 | 480 | >50 | >50 | >50 | >50 |
| 13 | 1.5 | 51 | 153 | >50 | >50 | >50 | >50 |

TABLE 4
Physicochemical properties of stapled ATSP-7041 C-terminal
peptides determined through various methods

| Compound | CD (% | | HPLC | Solubility (uM) |
|---|---|---|---|---|
| SEQ ID NO | helicity) | AlogP98 | LogD | (PBS, pH 7) |
| 1 | | 0.83 | 2.93 | 108 |
| 2 | | −0.53 | nd | nd |
| 3 | 47 | −0.53 | 3.15 | 157 |
| 4 | 45 | 2.58 | nd | nd |
| 5 | | 2.58 | 4.12 | 9 |
| 6 | | 1.02 | nd | nd |
| 7 | | 1.02 | 3.59 | 2 |
| 8 | 35 | 1.33 | nd | nd |
| 9 | | 1.12 | 2.93 | 153 |
| 10 | | −2.31 | 2.14 | 154 |
| 11 | | −4.17 | 2.25 | 163 |
| 12 | | −0.73 | 2.69 | 128 |
| 13 | 48 | 0.52 | 2.74 | 167 |

TABLE 5
Cellular proliferation activity of stapled DPMI d peptides
determined through various biochemical methods

| | | HCT116 | | p53 null Ca Ski |
|---|---|---|---|---|
| | HCT116 | proliferation | p53 null Ca Ski | proliferation |
| | proliferation EC₅₀ | % Max | proliferation EC₅₀ | % Max |
| SEQ ID | (uM, 72 h, 10% | (uM, 72 h, 10% | (nM, 72 h, 10% | (nM, 72 h, 10% |
| NO | serum) | serum) | serum) | serum) |
| 2 | 0.570 | 89 | 23.7 | 29 |
| 3 | 0.420 | 71 | >50 | 10 |
| 4 | 1.21 | 68 | | |
| 10 | 0.187 | 75 | >50 | −18% |
| 11 | 0.79 | 73 | >50 | −18% |
| 12 | 0.124 | 72 | >50 | 3 |
| 13 | 0.073 | 77 | >50 | 39 |

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

1. A crosslinked peptidomimetic macrocycle comprising:
(I)
(SEQ ID NO: 18)
R₁-R₂-betaAla-L-T-F-X₁-E-Y-W-A-Q-
R₃-X₂-Z₁-Z₂-Z₃-Z₄-Z₅-Z₆-R₄,
wherein:
R₁ is selected from acyl and C₁₋₁₂alkyl;
R₂ is a natural or non-natural L-amino acid residue;
R₃ is an aliphatic natural or non-natural amino acid residue;
R₄ is selected from —OH, —NH₂, and one to three L- or D-amino acid residues wherein the C-terminal tail is an acid or an amide group;
each of X₁ and X₂ is independently an α-monosubstituted or α,α-disubstituted non-natural L- or D-amino acid residue; and X₁ and X₂ are crosslinked from their respective alpha carbons via a hydrocarbon linkage; and
each of Z₁, Z₂, Z₃, Z₄, Z₅ and Z₆ is independently a natural or non-natural amino acid residue.

2. The crosslinked peptidomimetic macrocycle of claim 1, wherein R₁ is acetyl.

3. The crosslinked peptidomimetic macrocycle of claim 1, wherein R₂ is selected from a Lysine residue and an azido Lysine residue.

4. The crosslinked peptidomimetic macrocycle of claim 1, wherein R₃ is a cyclobutyl Alanine residue.

5. The crosslinked peptidomimetic macrocycle of claim 1, wherein R₄ is selected from —NH₂ and an L- or D-amino acid residue wherein the C-terminal tail is an amide.

6. The crosslinked peptidomimetic macrocycle of claim 1, wherein each of X₁ and X₂ is independently an α-monosubstituted or α,α-disubstituted non-natural L- or D-amino acid residue; and X₁ and X₂ are crosslinked from their respective alpha carbons via an alkene hydrocarbon linkage.

7. The crosslinked peptidomimetic macrocycle of claim 1, wherein each of X₁ and X₂ is independently an α-monosubstituted or α,α-disubstituted non-natural L- or D-amino acid residue; and X₁ and X₂ are crosslinked from their respective alpha carbons via a di-alkyne hydrocarbon linkage.

8. The crosslinked peptidomimetic macrocycle of claim 1, wherein each of X₁ and X₂ is independently selected from an (R)-2-amino-2-methyldec-9-enoic acid residue and an (S)-2-amino-2-methylhept-6-enoic acid residue; and X₁ and X₂ are crosslinked from their respective alpha carbons via an alkene linkage.

9. The crosslinked peptidomimetic macrocycle of claim 1, wherein each of X₁ and X₂ is independently selected from an (R)-2-amino-2-methyloct-7-ynoic acid residue and an (S)-2-amino-2-methylhept-6-ynoic acid residue; and X₁ and X₂ are crosslinked from their respective alpha carbons via a di-alkyne linkage.

10. The crosslinked peptidomimetic macrocycle of claim 1, wherein the Z₁-Z₂-Z₃-Z₄-Z₅-Z₆ sequence is selected from AAFAAF (SEQ ID NO: 16), AAFA(alpha-Me-Glu)F (SEQ ID NO: 19), AAEAA(D-Ala) (SEQ ID NO: 20), EAFAAF (SEQ ID NO: 21), AAAAAA (SEQ ID NO: 28) and AAAAA(D-Ala) (SEQ ID NO: 22).

11. The crosslinked peptidomimetic macrocycle of claim 10, wherein:
the Z₁-Z₂-Z₃-Z₄-Z₅-Z₆ sequence is selected from AAFAAF (SEQ ID NO: 16), AAFA(alpha-Me-Glu)F (SEQ ID NO: 19), EAFAAF (SEQ ID NO: 21) and AAAAA(D-Ala) (SEQ ID NO: 22).

12. The crosslinked peptidomimetic macrocycle of claim 1, wherein:
R₁ is acetyl;
R₂ is selected from a Lysine residue and an azido Lysine residue;
R₃ is a cyclobutyl Alanine residue;
R₄ is —NH₂;
each of X₁ and X₂ is independently an α-monosubstituted or α,α-disubstituted non-natural L- or D-amino acid residue; and X₁ and X₂ are crosslinked from their respective alpha carbons via an alkene or a di-alkyne hydrocarbon linkage; and
the Z₁-Z₂-Z₃-Z₄-Z₅-Z₆ sequence is selected from AAFAAF (SEQ ID NO: 16), AAFA(alpha-Me-Glu)F (SEQ ID NO: 19), AAEAA(D-Ala) (SEQ ID NO: 20), EAFAAF (SEQ ID NO: 21), AAAAAA (SEQ ID NO: 28) and AAAAA(D-Ala) (SEQ ID NO: 22).

13. The crosslinked peptidomimetic macrocycle of claim 1, wherein:
R₁ is acetyl;
R₂ is selected from a Lysine residue and an azido Lysine residue;
R₃ is a cyclobutyl Alanine residue;
R₄ is —NH₂;
each of X₁ and X₂ is independently selected from an (R)-2-amino-2-methyldec-9-enoic acid residue and an (S)-2-amino-2-methylhept-6-enoic acid residue; and X₁ and X₂ are crosslinked from their respective alpha carbons via an alkene linkage; and
the Z₁-Z₂-Z₃-Z₄-Z₅-Z₆ sequence is selected from AAFAAF (SEQ ID NO: 16), AAFA(alpha-Me-Glu)F (SEQ ID NO: 19), EAFAAF (SEQ ID NO: 21) and AAAAA(D-Ala) (SEQ ID NO: 22).

14. The crosslinked peptidomimetic macrocycle of claim 1, wherein:
R₁ is acetyl;
R₂ is selected from a Lysine residue and an azido Lysine residue;
R₃ is a cyclobutyl Alanine residue;
R₄ is —NH₂;
each of X₁ and X₂ is independently selected from an (R)-2-amino-2-methyloct-7-ynoic acid residue and an (S)-2-amino-2-methylhept-6-ynoic acid residue; and X₁ and X₂ are crosslinked from their respective alpha carbons via a di-alkyne linkage; and
the Z₁-Z₂-Z₃-Z₄-Z₅-Z₆ sequence is selected from AAFAAF (SEQ ID NO: 16), AAFA(alpha-Me-Glu)F (SEQ ID NO: 19), EAFAAF (SEQ ID NO: 21) and AAAAA(D-Ala) (SEQ ID NO: 22).

15. The crosslinked peptidomimetic macrocycle of claim 1, having formula (IV):

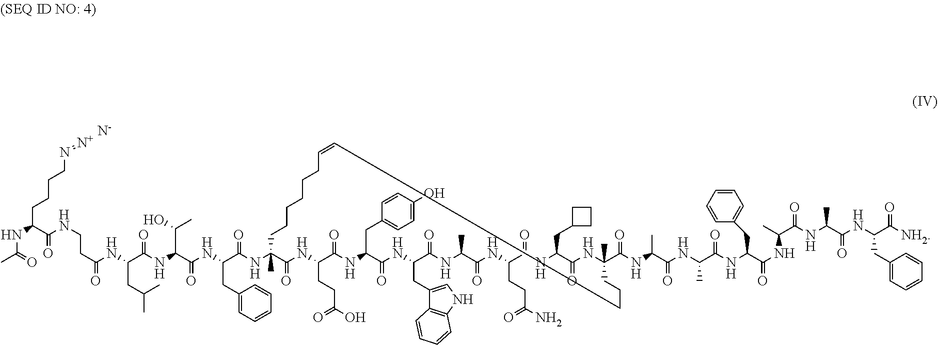

16. The crosslinked peptidomimetic macrocycle of claim 1, having formula (IX):

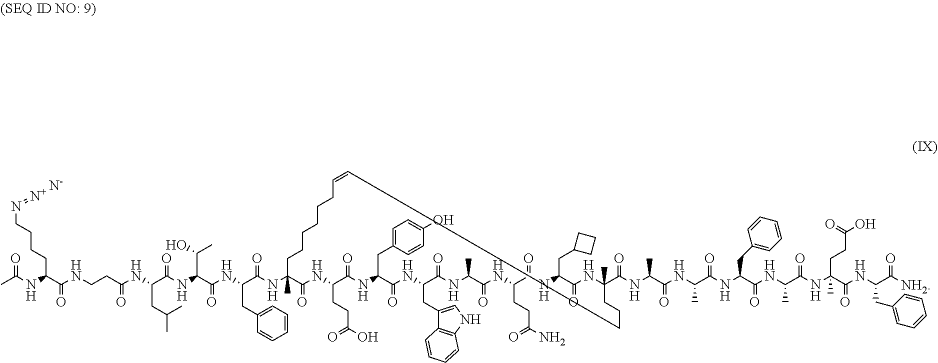

17. The crosslinked peptidomimetic macrocycle of claim 1, having formula (X):

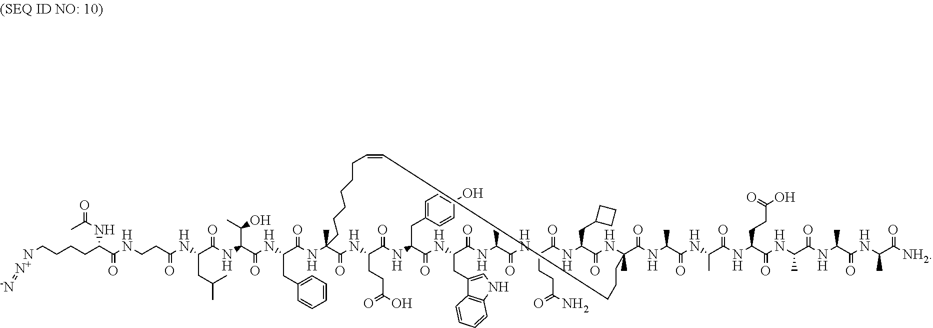

18. The crosslinked peptidomimetic macrocycle of claim 1, having formula XIII):

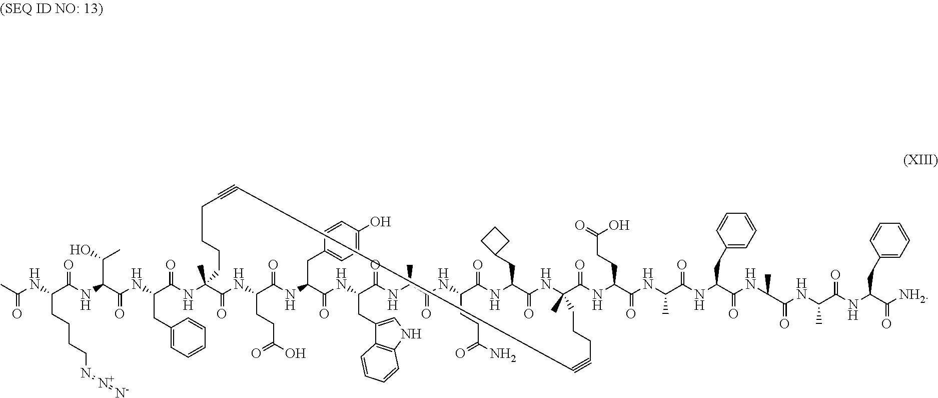 [SMILES: CC(=O)N[C@@H](CCCCN=[N+]=[N-])C(=O)N[C@H](C(=O)N[C@@H](Cc1ccccc1)C(=O)N[C@]1(C)CCCCC#CC#CCCC[C@@](C)(C(=O)N[C@@H](CCC(=O)O)C(=O)N[C@@H](C)C(=O)N[C@@H](Cc2ccccc2)C(=O)N[C@H](C)C(=O)N[C@@H](C)C(=O)N[C@@H](Cc2ccccc2)C(N)=O)NC(=O)[C@H](CC2CCC2)NC(=O)[C@H](CCC(N)=O)NC(=O)[C@H](C)NC(=O)[C@H](Cc2c[nH]c3ccccc23)NC(=O)[C@H](Cc2ccc(O)cc2)NC(=O)[C@H](CCC(=O)O)NC1=O)[C@@H](C)O]

19. The crosslinked peptidomimetic macrocycle of claim 1, wherein the peptidomimetic macrocycle binds both MDM2 and MDMX; is cell permeable without inducing detectable disruption to the cell membrane as determined by a lactate dehydrogenase (LDH) release assay; and activates p53 intracellularly.

20. A pharmaceutical composition comprising the crosslinked peptidomimetic macrocycle of claim 1 and a pharmaceutically acceptable carrier or excipient.

21. A method for treating cancer in a subject comprising administering to the subject a crosslinked peptidomimetic macrocycle of claim 1.

22. The method of claim 21, wherein the cancer is selected from the group consisting of melanoma, non-small cell lung cancer, head and neck cancer, urothelial cancer, breast cancer, gastrointestinal cancer, multiple myeloma, hepatocellular cancer, non-Hodgkin lymphoma, renal cancer, Hodgkin lymphoma, mesothelioma, ovarian cancer, small cell lung cancer, esophageal cancer, anal cancer, biliary tract cancer, colorectal cancer, cervical cancer, thyroid cancer, salivary cancer, pancreatic cancer, bronchus cancer, prostate cancer, pancreatic cancer, stomach cancer, ovarian cancer, urinary bladder cancer, brain or central nervous system cancer, peripheral nervous system cancer, uterine or endometrial cancer, cancer of the oral cavity or pharynx, liver cancer, kidney cancer, testicular cancer, biliary tract cancer, small bowel or appendix cancer, adrenal gland cancer, osteosarcoma, chondrosarcoma, and cancer of hematological tissues.

23. A method of modulating the activity of p53 and/or MDM2 and/or MDMX in a subject comprising administering to the subject a crosslinked peptidomimetic macrocycle of claim 1.

24. A method of antagonizing the interaction between p53 and MDM2 and/or between p53 and MDMX in a subject comprising administering to the subject a crosslinked peptidomimetic macrocycle of claim 1.

25. A pharmaceutical composition of a crosslinked peptidomimetic macrocycle of claim 1.

26. The pharmaceutical composition of claim 25, wherein the cancer is selected from the group consisting of melanoma, non-small cell lung cancer, head and neck cancer, urothelial cancer, breast cancer, gastrointestinal cancer, multiple myeloma, hepatocellular cancer, non-Hodgkin lymphoma, renal cancer, Hodgkin lymphoma, mesothelioma, ovarian cancer, small cell lung cancer, esophageal cancer, anal cancer, biliary tract cancer, colorectal cancer, cervical cancer, thyroid cancer, salivary cancer, pancreatic cancer, bronchus cancer, prostate cancer, pancreatic cancer, stomach cancer, ovarian cancer, urinary bladder cancer, brain or central nervous system cancer, peripheral nervous system cancer, uterine or endometrial cancer, cancer of the oral cavity or pharynx, liver cancer, kidney cancer, testicular cancer, biliary tract cancer, small bowel or appendix cancer, adrenal gland cancer, osteosarcoma, chondrosarcoma, and cancer of hematological tissues.

27.-28. (canceled)

29. A method of treatment for treating cancer comprising administering to a subject a therapeutically effective amount of a crosslinked peptidomimetic macrocycle of claim 1 and a therapeutically effective dose of a chemotherapy agent or radiation.

30. The method of treatment of claim 29, wherein the chemotherapy agent or radiation is administered to the subject followed by administration of the crosslinked peptidomimetic macrocycle; the peptidomimetic macrocycle is administered to the subject followed by administration of the chemotherapy agent or radiation; or the chemotherapy agent or radiation is administered to the subject simultaneously with administration of the peptidomimetic macrocycle.

31. A method of treatment for the treatment of a cancer comprising a therapeutically effective amount of a crosslinked peptidomimetic macrocycle of claim 1 and a therapeutically dose of a chemotherapy agent or radiation.

32. The method of treatment of claim 29, wherein the chemotherapy agent is selected from the group consisting of actinomycin, all-trans retinoic acid, alitretinoin, azacitidine, azathioprine, bexarotene, bleomycin, bortezomib, carmofur, carboplatin, capecitabine, cisplatin, chlorambucil, cyclophosphamide, cytarabine, dacarbazine, daunorubicin, docetaxel, doxifluridine, doxorubicin, epirubicin, epothilone, etoposide, fluorouracil, gemcitabin, hydroxyurea, idarubicin, imatinib, ixabepilone, irinotecan, mechlorethamine, melphalan, mercaptopurine, methotrexate, mitoxantrone, nitrosoureas, oxaliplatin, paclitaxel, pemetrexed, romidepsin, tegafur, temozolomide (oral dacarbazine), teniposide, tioguanine, topotecan, utidelone, valrubicin, vemurafenib, vinblastine, vincristine, vindesine, vinorelbine, and vorinostat.

33. A method of treatment for treating cancer comprising administering to a subject a therapeutically effective amount of a crosslinked peptidomimetic macrocycle of claim 1 and a therapeutically effective amount of a checkpoint inhibitor.

34. The method of treatment of claim 33, wherein the checkpoint inhibitor is an anti-PD1 antibody or an anti-PD-L1 antibody.

35. The method of treatment of claim 34, wherein the therapy further includes administering to the subject a therapeutically effective dose of a chemotherapy agent or radiation.

36. A method of treatment comprising administering to a subject having the cancer a vector comprising a nucleic acid molecule encoding a wild-type p53 protein or p53 variant with transcriptional activation activity followed by one or more administrations of a therapeutically effective amount of a crosslinked peptidomimetic macrocycle of claim 1.

37. The method of treatment of claim 36, wherein the vector is a plasmid, a retrovirus, adenovirus, or adeno-associated virus.

38. The method of treatment of claim 37, wherein the subject is administered a chemotherapy or radiation treatment prior to administering the vector to the subject or subsequent to administering the vector to the subject.

39. The method of treatment of claim 36, wherein the therapy further includes administering to the subject a checkpoint inhibitor.
